(19) United States
(12) Patent Application Publication
(10) Pub. No.: US 2003/0215792 A1
(43) Pub. Date: Nov. 20, 2003

(54) Nucleic acid molecule comprising a nucleic acid sequence coding for a chemokine, a neuropeptide precursor, or at least on neuropeptide

(75) Inventors: Hans Werner Muller, Duesseldorf (DE); Frank Bosse, Duesseldorf (DE); Mark Gleichmann, Tuebingen (DE); Clemens Gillen, Aachen (DE); Johannes Auer, Grafenaschau (DE)

Correspondence Address: MORGAN, LEWIS & BOCKIUS LLP, 1701 MARKET STREET, PHILADELPHIA, PA, 19103-2921, US

(73) Assignee: Hans Werner Mueller

(21) Appl. No.: 10/308,322

(22) Filed: Dec. 2, 2002

Related U.S. Application Data
(63) Continuation of application No. PCT/EP01/06250, filed on Jun. 1, 2001.

(30) Foreign Application Priority Data
Jun. 2, 2000 (DE) 100 27 383.1

(51) Int. Cl. C12Q 1/70; C12Q 1/68; G01N 33/53; G01N 33/567; C07H 21/04; C12P 21/02; A61K 39/395

(52) U.S. Cl. 435/5; 435/6; 435/7.2; 435/69.5; 435/320.1; 435/325; 424/145.1; 530/388.23; 536/23.5

(57) ABSTRACT

The invention concerns a nucleic acid molecule which includes a nucleic acid sequence coding for a chemokine, a neuropeptide precursor or at least one neuropeptide, as well as a host cell which contains this nucleic acid molecule. In addition the invention concerns a polypeptide molecule which functions as chemokine or neuropeptide or contains at least one neuropeptide, as well as fragments thereof which include at least one neuropeptide, and a procedure for the manufacture of the polypeptide molecule or of a fragment thereof. In addition the invention concerns antibodies, demonstration procedures and test-kits as well as pharmaceutical preparations.

The purpose on which present invention is based is to make new means available which can be put to use aimed at the diagnosis and/or treatment of diseases which are associated with a defect of the SDF-1 factor or its receptor (CXCR4).

The invention concerns a nucleic acid molecule which includes a nucleic acid sequence coding for a chemokine, a neuropeptide precursor, or at least one neuropeptide, as well as host cells containing this nucleic acid molecule. The invention in addition concerns a polypeptide molecule which functions as chemokine or neuropeptide or contains at least one neuropeptide, as well as fragments thereof which contain at least one neuropeptide, and a procedure for the manufacture of the polypeptide molecule or of a fragment thereof. Besides that the invention concerns antibodies, demonstration procedures and Test-kits as well as pharmaceutical preparations.

SDF-1α (stromal cell derived factor 1α) and its isoform SDF-1β arising from alternative splicing were originally cloned from a line of bone-marrow stroma cells of the mouse (Tashiro et al. 1993). On the basis of the established homology of the derived amino-acid sequence with the sequences of interleukin 8 (32%) and of the macrophage inflammation protein 1α (32%) and the presence of four characteristic cysteine residues, SDF-1α and SDF-1β have been assigned to the group of CXC (α) chemokines. The CXC(α) chemokines are a sub-group of the family of intercrine cytokines, which consists of various distantly related inflammation-promoting cytokines. The cDNA sequences for SDF-1α and SDF-1β of the mouse and of man display strong homology with each other and arise from alternative splicing of a single gene.

The biological function of SDF-1 was investigated with the aid of human SDF-1α. SDF-1α is required for the maturation of B-cells, operates T-lymphotropy and induces cell death in the neuronal cell line hNT. SDF-1α is a natural ligand of the CXCR4(LESTR/Fusin) chemokine receptor of T cells, which is a binding co-factor of T-lymphotropic HIV1 strains. SDF-1α and β-manifest both in vitro and in vivo a “growth-arrest”-specific expression pattern in fibroblasts and hepatocytes. Mice in which the SDF-1 gene has been inactivated display a reduced formation of B-cells, a defect of the ventricular septum and defects of cell migration into the cerebellum, and die shortly after birth. SDF-1 could play an important part in nerve regeneration.

The present invention has as its basis the intention of making new means available which are aimed at the diagnosis and/or treatment of diseases which are associated with a defect of the SDF-1 factor or its receptors (CXCR4).

By means of the invention this purpose is attained through a nucleic acid molecule comprising:

(1) a nucleic acid sequence coding for a chemokine, a neuropeptide precursor or at least one neuropeptide, selected from the following sequences:

(a) a nucleic acid sequence agreeing with SEQ ID NO:1;

(b) a nucleic acid sequence which codes for a polypeptide with an amino-acid sequence agreeing with SEQ ID NO:2;

(c) a nucleic acid sequence which is at least 60% identical with the sequence indicated in (a);

(d) a sequence which hybridizes with the opposing strand of the sequence indicated in (a) or which would hybridize taking into account degeneration of the genetic code;

(e) a derivative of one of the sequences indicated in (a) or (b), obtained through substitution, addition, inversion and/or deletion of one or more nucleotides, which codes for a chemokine, a neuropeptide precursor or at least one neuropeptide; or

(2) a complementary sequence to one of the nucleic acid sequences indicated in (a) to (e).

The concept “polypeptide” as subsequently used in the description also includes peptides or proteins constructed from 7 or more amino-acids.

The concept “chemokine” stands for a member of a family of relatively small proteins which on the basis of a characteristic arrangement of cysteine groups is divided into four sub-groups:

C, CC, CXC and CX₃C. The chemokines bind to specific receptors (Rollins, B. J. 1997). In connection with the present invention the concept “chemokine” applies specifically to members of the CXC chemokine family. It deals mainly with the chemokine as a polypeptide molecule which in a Ca-imaging experiment under the conditions described in Köller st al (2001) evoked a 1.5 to 10-fold rise in intracellular calcium concentration in primary astrocytes and/or neurones from the central nervous system of rats or humans.

Biologically active and physiologically important signal molecules with regulatory and modulatory functions in the nervous system are described as “neuropeptides”. The functional domains include among others neurotransmission, receptor modulation, alterations in electrophysiological properties of cell membranes and metabolic processes. Neuropeptides are synthesized by neurones and released mostly at the synapses (Siegel et al., 1989).

By “neuropeptide precursor” is understood a protein forerunner which is converted into an active neuropeptide through proteolytic splitting.

The nucleic acid sequence contained in the nucleic acid molecule in accordance with the invention can be a genomic DNA, cDNA or synthetic DNA, whereby under synthetic DNA sequences is understood those which also contain modified internucleoside bonds.

In connection with the nucleic acid sequence in accordance with the invention the expression “at least 60% identical ” refers to identity at the DNA level, which can be decided according to recognized procedures, e.g. computer-supported sequence comparisons (Altschul et al., 1990).

The expression “identity” recognized by the expert signifies the degree of relationship between two or more nucleic acid molecules as determined through the agreement between the sequences. The percentage of “identity” is indicated by the percentage of identical regions in two or more sequences taking into consideration gaps and other sequence particulars.

The identity of related nucleic acid molecules with one another can be determined with the help of recognized procedures. As a rule special computer programmes have the particular calculation-bearing algorithm requirements inserted. Preferred procedures for the determination of identity most nearly produce the greatest agreement between the sequences investigated. Computer programmes for the determination of identity between two sequences include the GCG programme package, comprehending GAP (Devereux, J., et al., Nucleic Acids Research 12 (12) 387, 1984), Genetics Computer Group university of Wisconsin, Madison Wis.; BLASTP, BLASTN and FASTA (Altschul et al., 1990) are however not restricted to these. The BLASTX programme can be obtained from the National Centre for Biotechnology Information (NCBI) and from other sources (BLAST handbook, Altschul S. et al., NCB NLM NIH Bethesda Md. 20894; Altschul et al., 1990). The well-known Smith Waterman algorithm can also be used for the determination of identity.

Preferred parameters for nucleic acid sequence comparison include those below:

| Algorithm: | Needleman and Wunsch (1970) |
|---|---|
| Comparison matrix: | Matches = +10 |
| | Mismatches = 0 |
| Gap penalty: | 50 |
| Gap length penalty: | 3 |

The GAP programme is also suitable for use with the foregoing parameters. The foregoing parameters are the standard parameters (default parameters) for nucleic acid sequence comparisons.

Further examples may be given of algorithms, gap opening penalties, gap extension penalties, comparison matrices named in the programme handbook, Wisconsin Package, Version 9, September 1997, which can be used. What is selected depends on the comparison being carried out and in addition on whether the comparison being carried out is between sequence pairs, for which GAP or Best Fit are preferred, or between a sequence and a comprehensive data bank, for which FASTA and BLAST are preferred. An agreement of 60% ascertained with the abovementioned algorithm is taken in the framework of this announcement to be 60% identity. Higher degrees of identity have corresponding validity.

The passage “sequence which hybridizes with the opposing strand of the sequence indicated in (a)” refers to a sequence which under stringent conditions hybridizes with the opposing strand of the sequence indicated under (a). For example, the hybridization might be carried out at 42° C. with a hybridization solution consisting of 5×SSPE, 5×Denhardt's, 0.1% SDS, 100 μg/ml salmon sperm DNA, 30-50% formamide (Sambrook et al., 1989). For the washing stage a twice-repeated 10-15 minute washing in 2×SSPE, 0.1% SDS at 42° C., followed by a twice-repeated 20 minute washing in 2×SSPE, 0.1% SDS at 50° C. Alternatively SSC may be used instead of SSPE in the washing solution.

Surprisingly, it was now found that the nucleic acid molecule in accordance with the discovery represents a new member of the SDF family of chemokines, and is consequently referred to as SDF-1γ. The cloning and characterizing of SDF-1γ-cDNA as well as the nucleic acid sequence and the amino-acid sequence derived from it for the human SDF-1γ and the SDF-1γ of rats are described in the Examples.

The SDF-1γ nucleic acid sequence consists of the complete nucleic acid sequence of SDF-1β and an additional sequence of 2572 nucleotides which in the same downstream reading-frame joins codon 89 of SDF-1β. Through this insert there results for the new SDF-1γ polypeptide an amino-acid sequence with 119 amino-acids and a theoretical molecular weight of 13.6 Kd, in which the sequence at the carboxy terminal is lengthened by 30 amino-acids in comparison with the. known SDF-1α sequence. It is suspected that SDF-1γ results from the insertion of a new alternative exon IIIa between the known exons III and IV (cf. Shirozu et al., 1995).

In the region of the carboxy terminal of the amino-acid sequence of SDF-1γ 5 groups of two basic amino-acids (Lys-Lys, Arg-Arg, and Lys-Arg) may form the recognition pattern for a membrane-bound protease of the Golgi system and secretory vesicle. Through proteolytic splitting at this site five short peptides (SEQ ID NO:5, SEQ ID NO:6, SEQ ID NO:8, SEQ ID NO:9, SEQ ID NO:10) are created and a shortened protein (SEQ ID NO:7) from which two peptides and one polypeptide (SEQ ID NO:5, SEQ ID NO:6 and SEQ ID NO:7) constitute a carboxy-terminal glycin residue. Peptides with a glycin residue at the carboxy terminus are potential substrates for the peptidyl-α-amidizing monoxygenase (PAM) which catalyses the carboxy-terminal splitting of carboxylate whereby result the α-amidized carboxy termini (CONH₂) which are characteristic of neuropeptides (see review by Eipper et al., 1992).

The rat SDF-1β transcript (see SEQ OD NO:16) codes for a protein with 93 amino-acids (see SEQ ID NO:17) and a theoretical molecular weight of 10.5 kD, in which the first 19 amino-acids represent a signal sequence for secreted proteins. The first 17 amino-acids of the mature protein, which occur in all isoforms, are necessary for binding to the CXCR4 receptor (cf. Loetscher et al. 1998, Doranz et al. 1999). Two basic amino-acids (lys 89 and arg 90) in the carboxy-terminal region provide a recognition pattern for the proteolytic splitting, from which a pentapeptide (lys 89-met 93, SEQ ID NO:18) and a shortened protein are produced.

In a practical form of the invention the nucleic acid molecule according to the invention contains a nucleic acid sequence which is at least 80%, preferably at least 90%, especially favourably at least 95%, identical with the nucleic acid sequence agreeing with SEQ ID NO:1.

Nucleic acid molecules which include a nucleic acid sequence agreeing with SEQ ID NO:3 or a polypeptide with an amino-acid sequence agreeing with a SEQ ID NO:4 coding nucleic acid sequence are especially preferred.

The nucleic acid molecule according to the invention may furthermore include a promotor suitable for expression, whereby the coding nucleic acid sequence remains under the control of the promotor. A “promotor suitable for expression” as used here signifies a DNA fragment through which the initiation point and the initiation frequency of the transcription (RNA synthesis) of a nucleic acid sequence, remaining under the control of the promotor element, which codes for a chemokine, a neuropeptide precursor, or at least a neuropeptide, are established in the host organism. The choice of promotor depends on the expression system used for the expression. In general constituent promoters are preferred, but inducible promotors, such as for instance the metallothioneine promotor, are also possible. Promotors worth considering for carrying out the invention include among others the FMD-, MOX-, TPS1-, PMA1- and DAS-promoters from Hansenula polymorpha, the ADH1-, PDC1-, GAP1- and CUP1-promoters from S. cerevisiae, the AXDH- and ASHB4-promoters from Arxula adeninovorans and the NDK1- and CPC2-promotors from Sordaria macrospora.

The nucleic acid molecule according to the invention may in addition also contain sequences of a vector which potentiate the replication of the nucleic acid molecule in a host cell and/or the integration of the nucleic acid molecule into the genome of a host cell. In the present state of the art numerous cloning and expression vectors are known, cf Recombinant Gene Expression Protocols, Meth. Mol. Biol. Vol 62, Humana Press, Hew Jersey, USA. For replication in a host cell the vector used must contain a replication initiation and if necessary further regulatory regions. The vector can be chosen from bacteriophages such as λ-derivatives, adenoviruses, plasmids, vaccinia viruses, baculoviruses, SV40 virus, retroviruses, plasmids such as Ti plasmids from Agrobacterium tumefasciens YAC- and BAC-vectors.

The object of the present invention is furthermore a host cell containing at least a nucleic acid molecule according to the invention, of which the host cell is a prokaryotic or eukaryotic cell suitable for the expression of the nucleic acid molecule and if necessary the processing of the resulting polypeptide molecule. In the present state of the art countless prokaryotic and eukaryotic expression systems are known. Host cells may be chosen for example from prokaryotic cells such as E. coli or B. subtilis, or from eukaryotic cells such as fungal cells, plant cells, insect cells and mammalian cells, e.g. CHO cells, COS or HeLa cells or derivatives thereof. In the present state of the art certain CHO production lines, for instance, are known, whose glycosylation patterns are altered in comparison with CHO cells. The eukaryotic cells are preferably the yeast Saccharomyes cerevisiae, the methylotrophic yeast Hansenula polymorpha, the dimorphic yeast Arxula adeninivorans or the filamentous fungus Sordania macrospora.

Furthermore the invention makes available a polypeptide molecule comprising an amino-acid sequence chosen from the following sequences:

(i) an amino-acid sequence which contains one of the amino-acid sequences agreeing with SEQ ID NO:5, SEQ ID NO:6, SEQ ID NO:7, SEQ ID NO:8, SEQ ID NO:9 and/or SEQ ID NO:10 and/or a combination of two or more of these sequences;

(ii) an amino acid sequence agreeing with SEQ ID NO:4;

(iii) an amino-acid sequence which corresponds to the sequence of amino-acid 20 to amino-acid 119 in SEQ ID NO:4;

(iv) an amino-acid sequence agreeing with SEQ ID NO:22;

(v) an amino-acid which is at least 85% identical with the sequences indicated in (i), (ii), (iii) or (iv).

In this connection the expression “at least 85% identical” refers to agreement at the level of the amino-acid sequence which be determined by means of recognized procedures, e.g. computer-generated sequence comparisons (Altschul et al., 1999).

The expression “identity” here signifies the degree of relationship between two or more nucleic acid molecules as determined through the agreement between the sequences, in which under agreement both identical agreement and conservative amino-acid replacement is to be understood. The percentage of “identity” is indicated by the percentage of identical regions in two or more sequences taking into consideration gaps and other sequence particulars.

The concept “conservative amino-acid replacement” refers to a replacement of one amino-acid residue by another amino-acid residue, in which the replacement should exert the most limited possible influence-on the (spatial) structure of the polypeptide molecule. Fundamentally four physico-chemical groups are distinguished, into which the naturally occurring amino-acids are divided. Arginine, lysine and histidine belong to the basic amino-acid group. To the acidic amino-acids belong glutamic acid and aspartic acid. The chargeless/polar amino-acids consist of glutamine, asparagine, serine, threonine and tyrosine. The non-polar amino-acids comprise phenylalanine, tryptophane, cysteine, glycine, alanine, valine, methionine, isoleucine, leucine and proline. In this context a conservative amino-acid replacement means the replacement of an indicated amino-acid by an amino-acid belonging to the same physico-chemical group.

The identity of polypeptide molecules related to one another can be determined with the aid of recognized procedures. Preferred procedures for the determination of identity lead most closely to the greatest agreement between the sequences investigated. Computer programmes for the determination of identity between two sequences include the GCG programme package, comprehending GAP (Devereux, J., et al., Nucleic Acids Research 12 (12) 387, 1984), Genetics Computer Group University of Wisconsin, Madison Wis.; BLASTP, BLASTN and FASTA (Altschul et al., 1990), but are not restricted to these. The BLASTX programme can be obtained from the National Centre for Biotechnology Information (NCBI) and from other sources (BLAST handbook, Altschul S. et al, NCB NLM NIH Bethesda Md. 20894; Altschul et al., 1990). The well-known Smith Waterman algorithm can also be used for the determination of identity.

Preferred parameters for sequence comparison include those below:

| Algorithm: | Needleman and Wunsch (1970) |
|---|---|
| Comparison matrix: | BLOSUM 62 of Henikoff and |
| | Henikoff (1992) |
| Gap penalty: | 12 |
| Gap length penalty: | 4 |
| Resemblance threshold value: | 0 |

The GAP programme is also suitable for use with the foregoing parameters. The foregoing parameters are the standard parameters (default parameters) for nucleic acid sequence comparisons, by which gaps at the ends do not lessen the identity value. With very short sequences it may be additionally necessary when comparing to a reference sequence to raise the expected value up to 100,000 and to reduce the word size to 2.

Further exemplary algorithms, gap opening penalties, gap extension penalties, and comparison remplates including that named in the programme handbook, Wisconsin package, version 9, September 1997, may be used. The choice is dependent on the comparison being carried out and in addition on whether the comparison is being done between two sequence pairs, for which GAP or Best Fit are preferred, or between a sequence and a comprehensive data-bank, for which FASTA or BLAST are preferred.

An agreement of 85% obtained with the abovenamed algorithm is, in the framework of this application, taken as 85% identity. Higher grades of identity are correspondingly valid.

In a practical form of the invention the polypeptide molecule according to the invention contains a sequence which is at least 90%, preferably at least 95%, identical with the amino-acid sequence indicated in the foregoing (i), (ii) (iii) or (iv). Especially preferred are polypeptide molecules which contain an amino-acid sequence agreeing with SEQ ID NO:12 or SEQ ID NO:13.

In a practical form of the invention the polypeptide molecule according to the invention contains the nucleic acid sequences agreeing with SEQ ID NO:5, SEQ ID NO:6 and/or SEQ ID NO:7. Preferred are polypeptide molecules according to the invention which contain the amino-acid sequences agreeing with SEQ ID NO:5 and SEQ ID NO:6.

In a further practical form the invention makes available a fusion protein comprising at least one polypeptide according to the invention.

Fragments of the polypeptide molecules according to the invention, which contain at least one neuropeptide, are similarly included in the invention. Fragments are preferred which contain at least one of the amino-acid sequences agreeing with SEQ ID NO:5, SEQ ID NO:6, SEQ ID NO:7, SEQ ID NO:8, SEQ ID NO:9 and/or SEQ ID NO:10. Especially preferred are fragments with the amino-acid sequence agreeing with SEQ ID NO:5, SEQ ID NO:6 or SEQ ID NO:7. The fragments of the polypeptide molecules according to the invention may also be modified, for example through glycosilation, phosphorylation, acetylation or amidization.

Preferably it is suggested that the peptide molecule according to the invention, fusion protein or fragment around a peptide molecule which in a Ca-imaging experiment under the conditions described in Köller (2001) produces a 1.5-10 fold rise in the intracellular calcium concentration in primary astrocytes and/or neurones from the central nervous system of rats or humans.

The object of the invention is in addition a procedure for the manufacture of a polypeptide molecule according to the invention and/or a fragment thereof, which includes the cultivation of a host cell in accordance with the invention under conditions suitable for the expression and possible processing and if necessary purification of the polypeptide molecule or fragment expressed. Alternatively the polypeptide molecules and fragments thereof may also be obtained through chemical and enzymatic synthesis such as for example Merrifield synthesis and/or fragment condensation. Combinations of chemical, enzymatic and recombinant manufacturing procedures come similarly to mind.

A further object of the invention is an antibody which is specific for the a polypeptide molecule according to the invention and/or a fragment thereof. Generally specific antibodies are producible through immunization of experimental animals, such e.g. as mice or rabbits, with the molecules or fragments in accordance with the invention, preferably bound to suitable high-molecular carrier molecules (often proteins). In this way the immunization can be facilitated by suitable state-of-the-art known adjuvants. Monoclonal antibodies are as usual obtainable through fusion of splenic cells taken from an immunized mouse with tumour cells and selection of the resulting hybridomata. The hybridomata, which secrete specific antibodies efficiently, may be decided upon by scanning those which remain. Alternatively antibodies nay be manufactured recombinantly; in the manufacture of recombinant antibodies the mRNA from hybridoma cells or B-lymphocytes is isolated and functions as the basis for the synthesis of the corresponding cDNA and is amplified by PCR. Following ligation into a suitable vector and insertion into a suitable host cell the antibody can be recovered from the cell culture residue or cell lysate. Recombinant antibodies permit a “humanization” of the antibody and are consequently less immunogenic. The relevant procedure is recognized as start-of-the-art.

For the demonstration of a polypeptide molecule according to the invention and/or fragment thereof in a biological specimen the invention provides an in vitro procedure which includes the bringing of the specimen into contact with a reagent specific for the polypeptide molecule and/or a fragment thereof and the demonstration of binding.

The invention in addition makes available a test-kit for the demonstration of a polypeptide or a fragment thereof, which contains at least a reagent specific for the polypeptide and/or fragment according to the invention. An example of such specific reagents are antibodies, antibody fragments, e.g. Fab or F(ab)₂ fragments or antibody derivatives, among which antibodies are especially preferred. The antibodies, antibody fragments, e.g. Fab or F(ab)2 fragments or antibody derivatives may be of monoclonal or polyclonal origin.

In addition the invention makes available an in vitro procedure for the demonstration in a biological specimen of a nucleic acid coding for a polypeptide molecule according to the invention, which includes:

the bringing into contact of the specimen with a nucleic acid molecule according to the invention and/or a fragment thereof, by which the nucleic acid and/or fragment bear a demonstrable token, and

the demonstration of the token

The object of the invention is in addition pharmaceutical preparations which contain at least a polypeptide molecule and/or fragment thereof in accordance with the invention and/or a pharmaceutically tolerable salt of such a polypeptide molecule or fragment. These pharmaceutical preparations may contain a pharmaceutically tolerable excipient and/or diluent. Suitable excipients or diluents are start-of-the-art recognized. Pharmaceutical preparations suitable for intravenous, subcutaneous or intramuscular administration are preferred. In a practical form of the pharmaceutical preparations according to the invention the preparation also contains besides one or more polypeptide molecules or fragments of the same according to the invention at least least one antibody.

In accordance with the invention these pharmaceutical preparations may be employed for the therapy of demyelinating or neurodegenerative diseases or of developmental disorders of the nervous system. A further use for pharmaceutical preparations in accordance with the invention is directed towards the prevention or treatment of an HIV infection and in particular an HIV encephalopathy in humans. The invention similarly embraces the use of pharmaceutical preparations in accordance with the invention for the therapy of diseases of the haemopoietic system, the immune system and the heart and circulatory system.

Pharmaceutical preparations containing at least one reagent specific for a polypeptide molecule according to the invention and/or a fragment thereof are also included in the invention. The reagent is preferably an antibody . Such pharmaceutical preparations may be employed in the diagnosis or therapy of demyelinating or neurodegenerative diseases or developmental disorders of the nervous system. A further use concerns the diagnosis or treatment of an HIV infection and in particular an HIV encephalopathy in humans

The following figures and examples elucidate the invention:

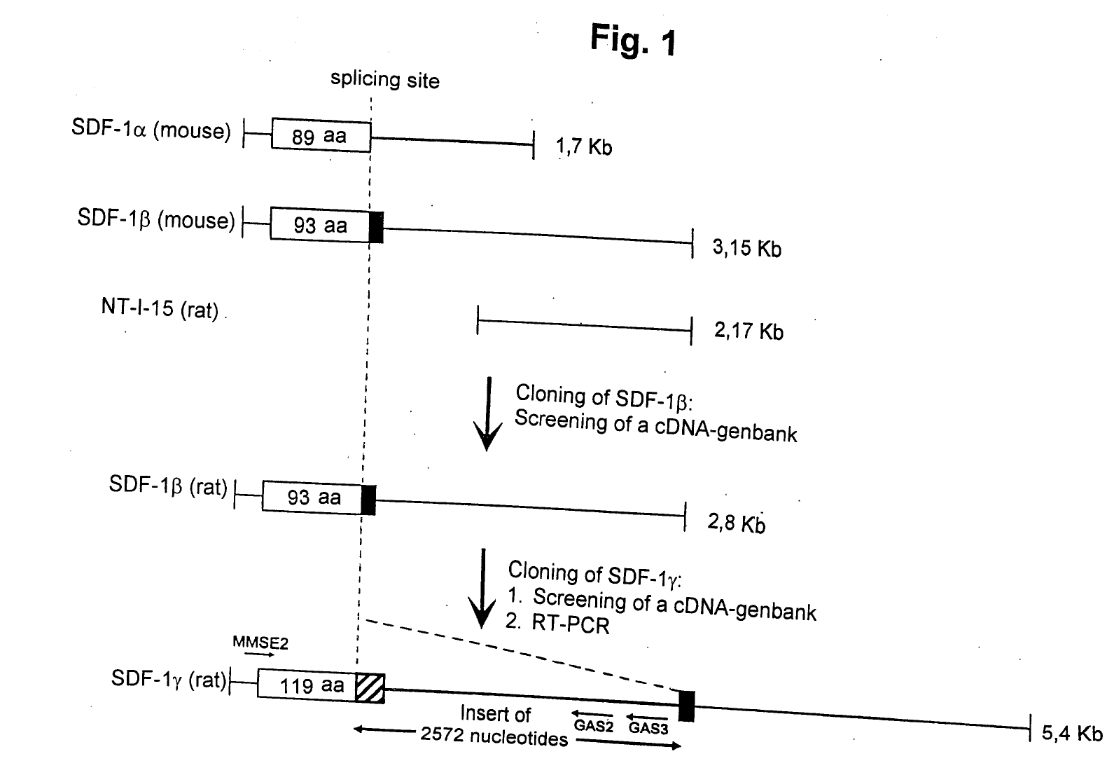

FIG. 1: shows the RT-PCR-based strategy for the cloning of SDF-1β- and SDF-1-γ-cDNA from rats. 5′ and 3′ UTR regions are represented by lines, coding regions by little boxes. Small arrows indicate the position and orientation of the primer. Identical or homologous sequences are represented by identical graphic elements. The last four codons of the coding region of SDF-1β are represented as small black boxes. The dashes stand for an insert of 2572 nucleotides in SDF-1γ. The 30 carboxyterminal codons of SDF-1γ are represented by small hatched boxes.

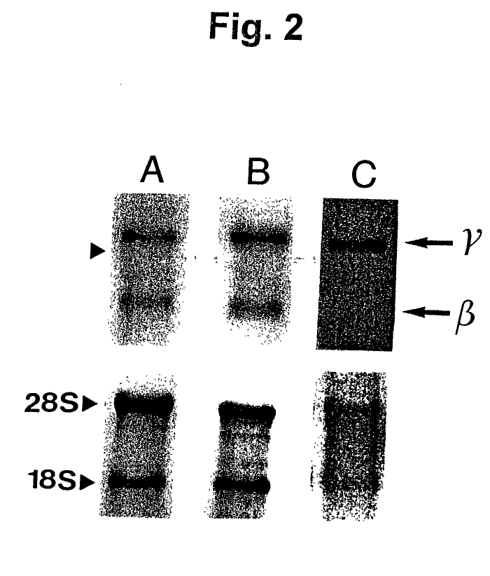

FIG. 2 shows the result of a Northern Blot test to demonstrate SDF-1β and SDF-1γ transcripts in the sciatic nerve of adult rats. The filters were hybridized (A) with a radioactively labelled PCR fragment of the NT-I-15-cDNA, 626 nucleotides long, which corresponds to the nucleotides 743-1368 of SDF-1β and is part of the 3′ UTR sequence common to SDF-1β and SDF-1γ, (B) with a PCR fragment, 190 nucleotides long, of the common coding region of all the SDF-1 isoforms, which corresponds to nucleotides 49-239 in the SEQ ID NO:16, (C) with a fragment 1702 nucleotides long which corresponds to the nucleotides 625-2327 of the SDF-1γ-cDNA and arose from the SDF-1γ specific insert through digestion of the PCR product obtained with the primers GAS2 and MMSE2 with Pvull.

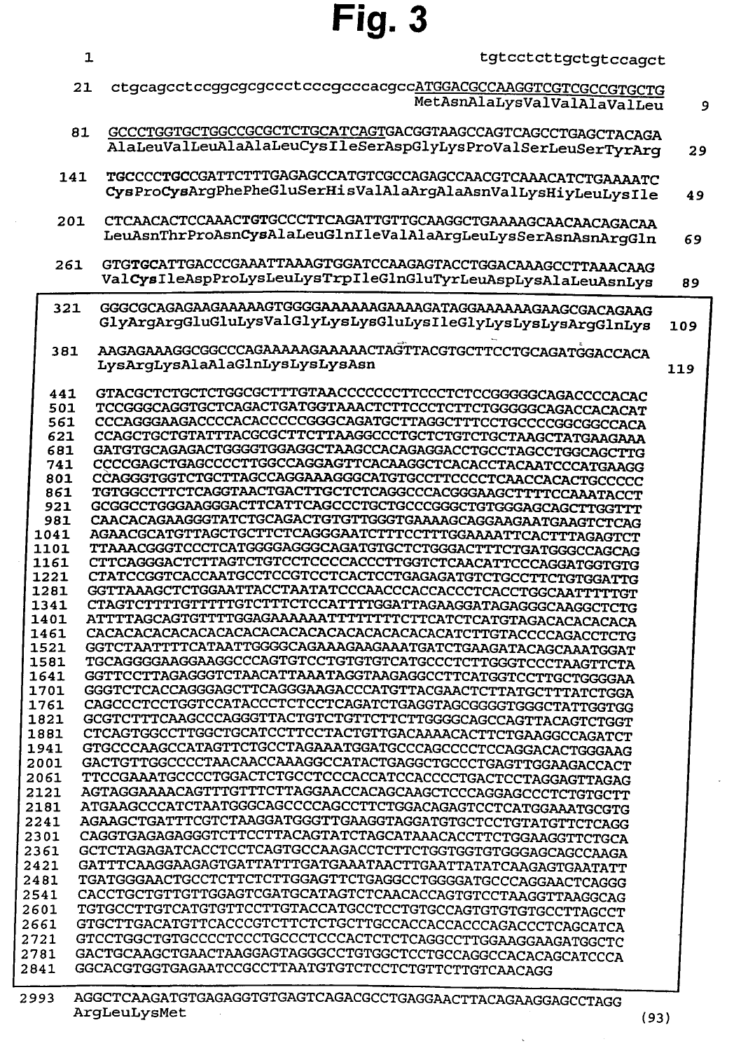

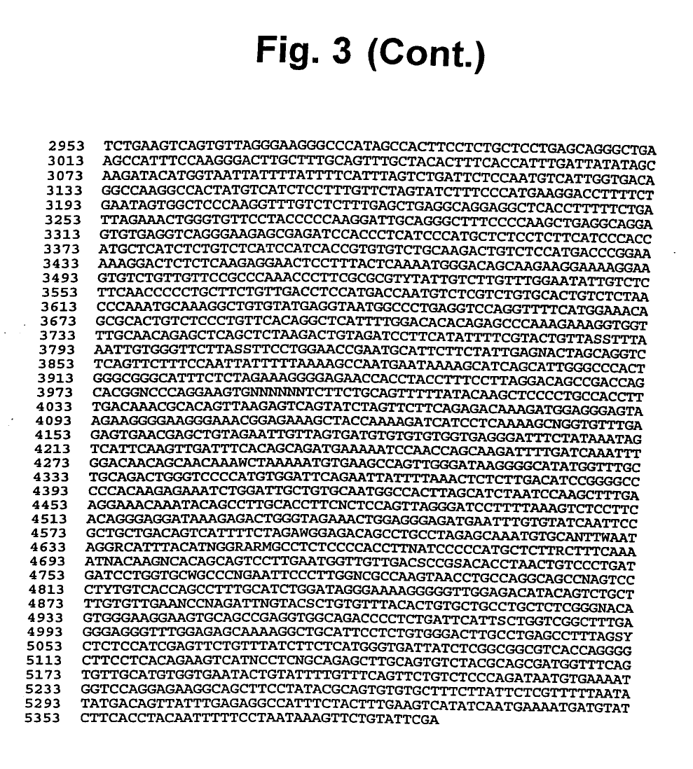

FIG. 3 shows the nucleic acid sequences from rats as well as the amino-acid sequences of the SDF-1β- and SDF-1γ-cDNA derived from them. The SDF-1γ-specific insert is thrown into relief by a frame. The nucleic acid sequence of the common signal peptide is underlined. The numbering of the nucleotides (left) and the amino-acids (right) correspond to the sequence of SDF-1γ. (93) characterizes the last amino-acids of SDF-1β

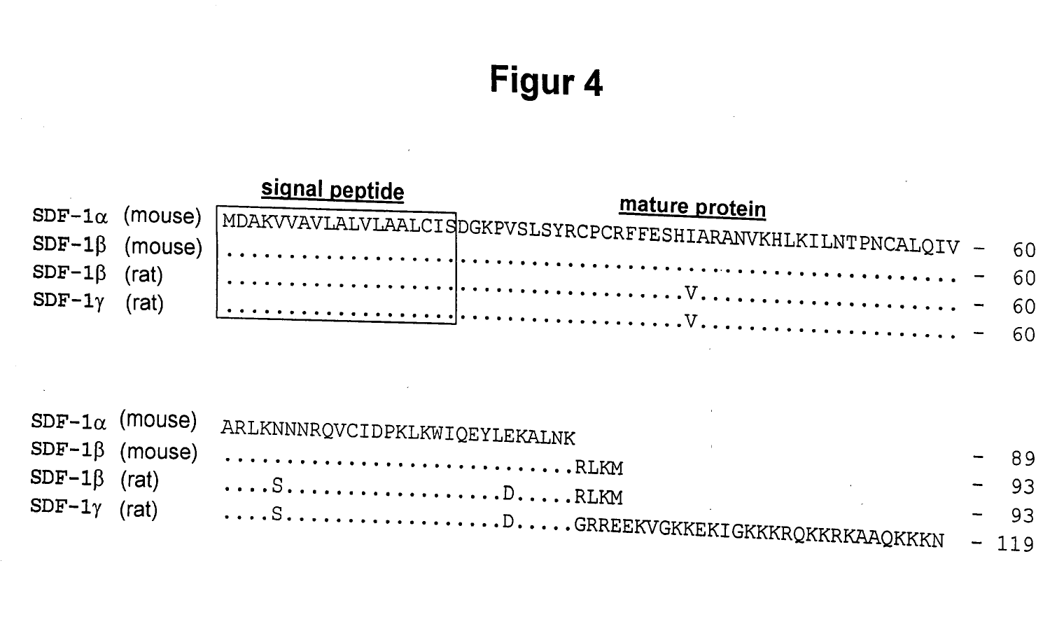

FIG. 4 shows a comparison of the amino-acid sequences of the SDF-1 proteins of mouse and rat. The points stand for identical amino-acids. The 19-amino-acid-long signal peptide is framed.

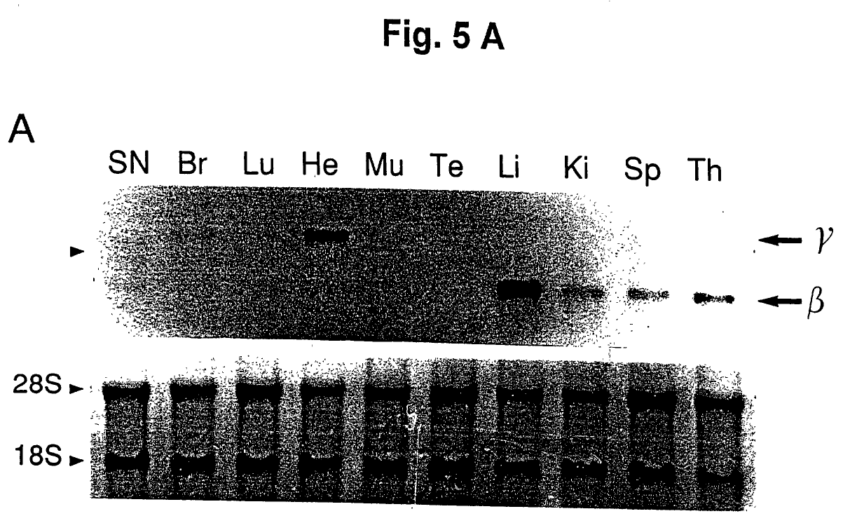

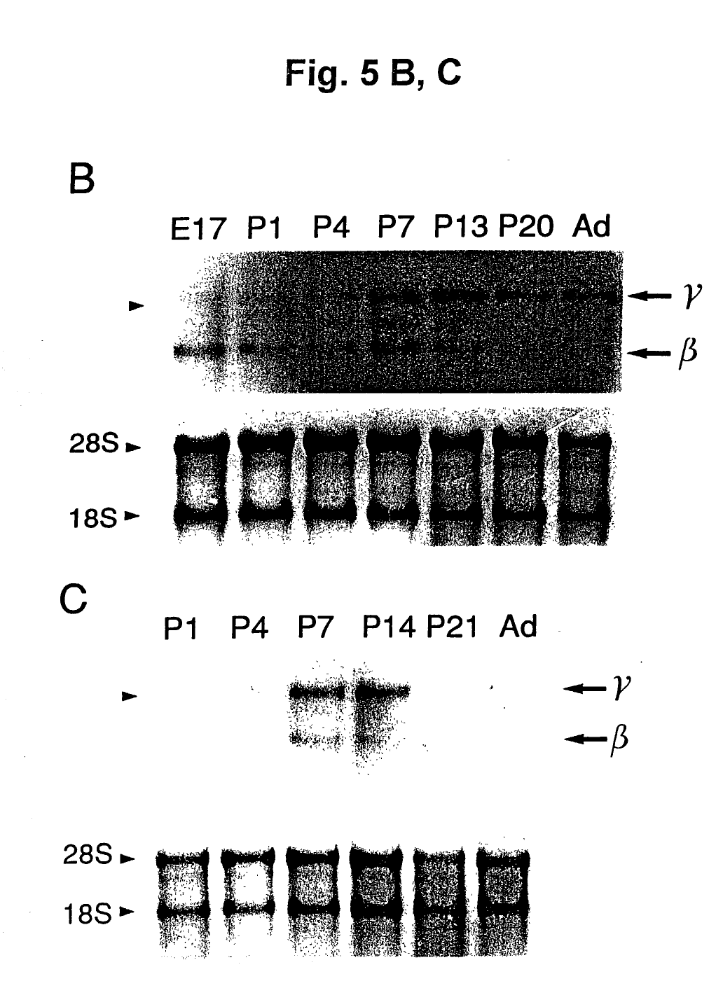

FIG. 5 shows the result of various Northern Blot tests for evidence of SDF-1β and SDF-1γ transcripts in various tissues (A) and stages of development (B). (A) Northern Blot filter with total RNA from the sciatic nerve (SN), brain (Br), lungs (Lu), heart (HE), muscle (Mu), testicles (Te), Liver (Li), kidneys (Ki), spleen (Sp) and thymus (Th) was hybridized with a radioactively labelled cDNA probe from the SDF-1β and SDF-1γ common 3′-UTR region. (B) Demonstration of SDF-1β and SDF-1γ mRNA in the brain of rats during development. Northern Blot filter with total RNA from the brain of 17-day-old rat embryos (E 17) as well as from rats 1, 4, 7, 13 and 20 days after birth (P1-20) and from adult animals (Ad) were hybridized as in (A). (C) Demonstration of SDF-1β-and SDF-1γ mRNA in the sciatic nerve of rats in process of development. Northern Blot filter with total RNA derived from the nerves of 1, 4, 7, 14 and 21-year-old rats (P1-21) and from adult rats (Ad) were hybridized with an SDF-1β/γ probe as in (A).The points of the arrows in the upper part indicate the position of the ribosomal 28S RNA; the lower parts show up with methylene blue-dyed Northern Blot filter.

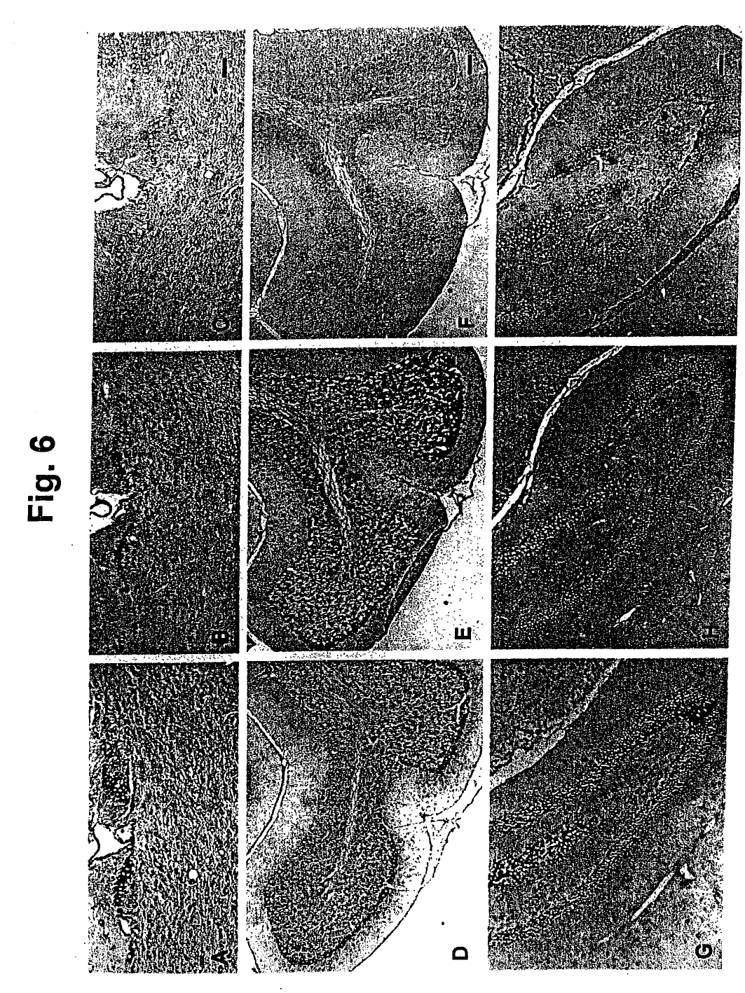

FIG. 6 shows the result of in situ hybridization tests for the cellular localization of SDF-1γ-mRNA in the brain of adult rats. The sections were incubated with a digoxigenin-UTP labelled 596-nucleotide-long antisense transcript derived from a sub-clone of all SDF-1 isoform total 5′ UTR and coding sequence (A, D, E) or from a sub-clone of the SDF-1γ specific insert (B, E, H). The hybridizing with sense-transcripts served as negative control (C, F, I). (A, B, C) Corpus callosum with “pearl-necklace”-like tracing of labelled oligodendrocytes and strongly labelled neurones in the bilateral slides of the indusium griseum dorsale of the corpus callosum. (D, E, F) Strong hybridization signals are observed in Purkinje and granular cells of the cerebellum and weak signals in the other slides. (G, H, I) Very strong hybridization signals are detected in the pyramidal and granular cells of the hippocampus. Line: 10 μm

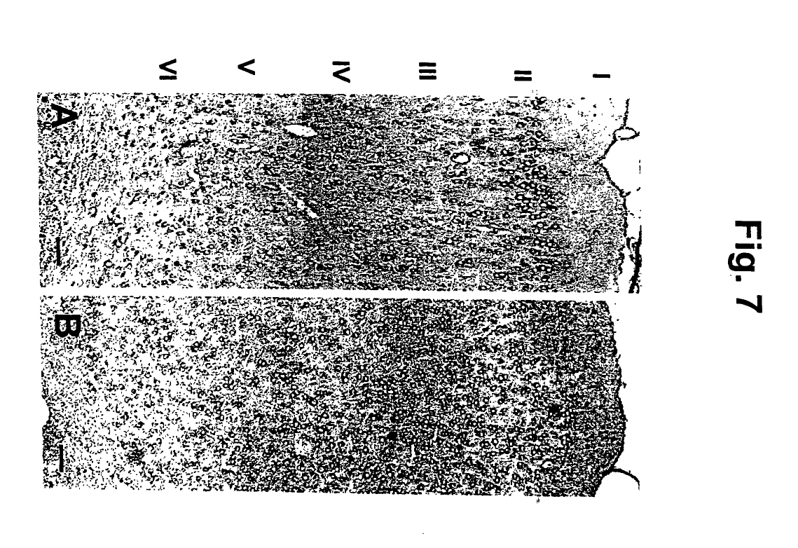

FIG. 7 shows the result of in situ hybridization tests for the cellular localization of SDF-1γ-mRNA in the neocortex of rats while using the same probe as in FIG. 6. With sections from the frontolateral (A) and mediolateral (B) regions of the neocortex the neurones in all of the neocortex slides (I-VI) are strongly labelled both with the antisense probe (A) common to all the SDF-1 mRNA isoforms and also with the SDF-1γ-specific probe.

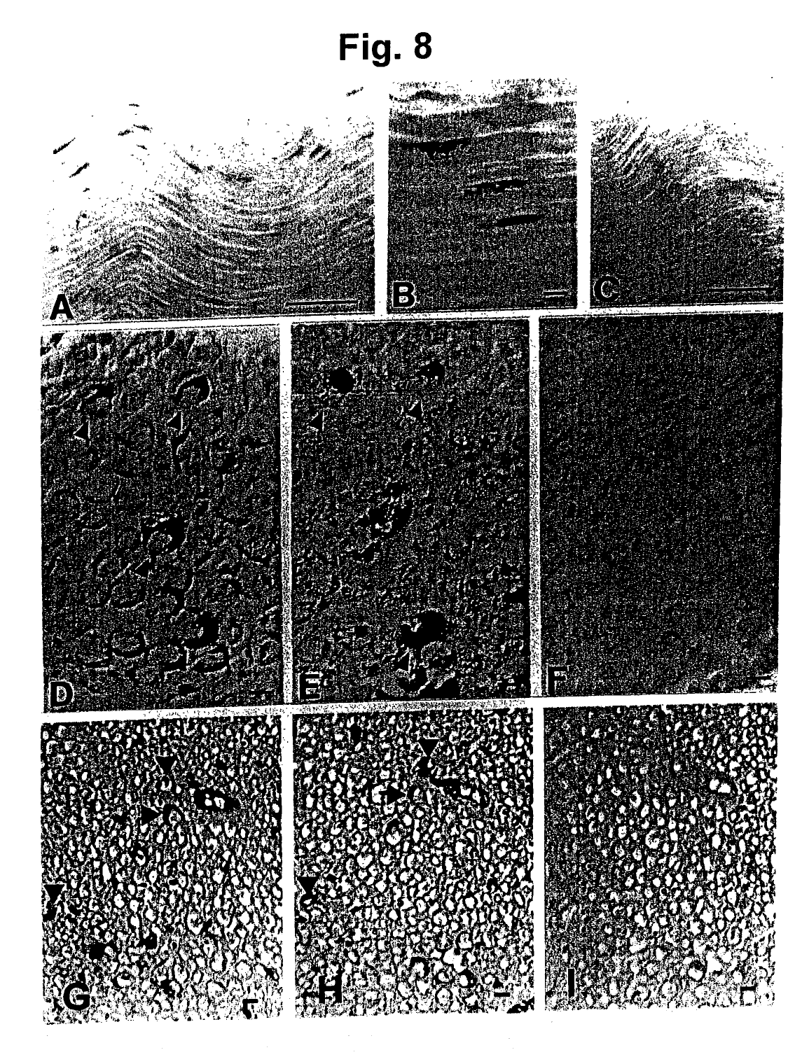

FIG. 8 shows the result of in situ hybridization tests for the cellular localization of SDF-1γ-mRNA in the sciatic nerve of adult rats. The sections were hybridized with digoxigenin-UTP-labelled RNA probes in sense and antisense orientation, which were derived (a) from the 3′ UTR region common to SDF-1β and SDF-1γ (A-C, E, F) (b) from the 5′-UTR and coding regions common to all SDF-1 isoforms (G) and from the SDF-1γ-specific insert. (H, I). (A, B, C) The longitudinal section shows more spindle-shaped Schwann cells in the neighbourhood of the axons. (D) A transverse section immune-dyed with an antibody against the S100 protein (a marker for Schwann cells). (E) The hybridization signals in a transverse section neighbouring the transverse section in (D) show labelled semicircular Schwann cells enclosing the axons which appear at the same place as the cells immunopositive for S100 (arrow points in D, E) (G, H) With either one of both antisense-transcript-labelled neighbouring transverse sections, which show numerous semicircular Schwann cells (see the arrow-points) and the wall of a blood-vessel in the upper right-hand corner. (C, F, I) Hybridization with transcripts in sense orientation served as a negative control. Lines in A, C, G-I: 100 μm, in D-F 10 μm.

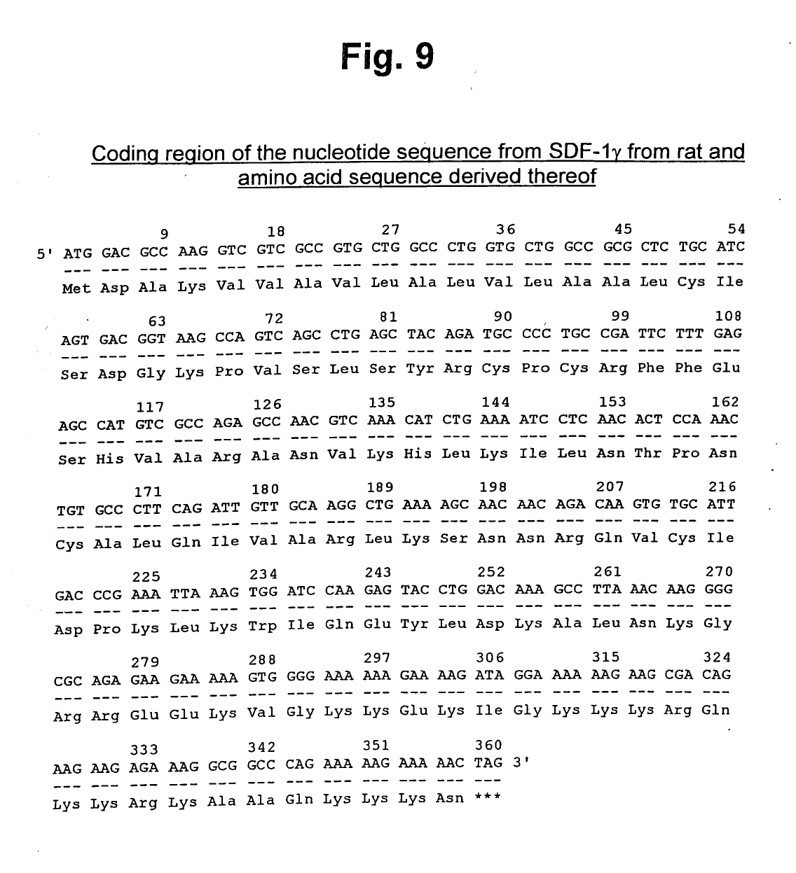

FIG. 9 shows the coding region of the nucleic acid sequence of SDF-1γ from rats and the amino acid sequence derived therefrom.

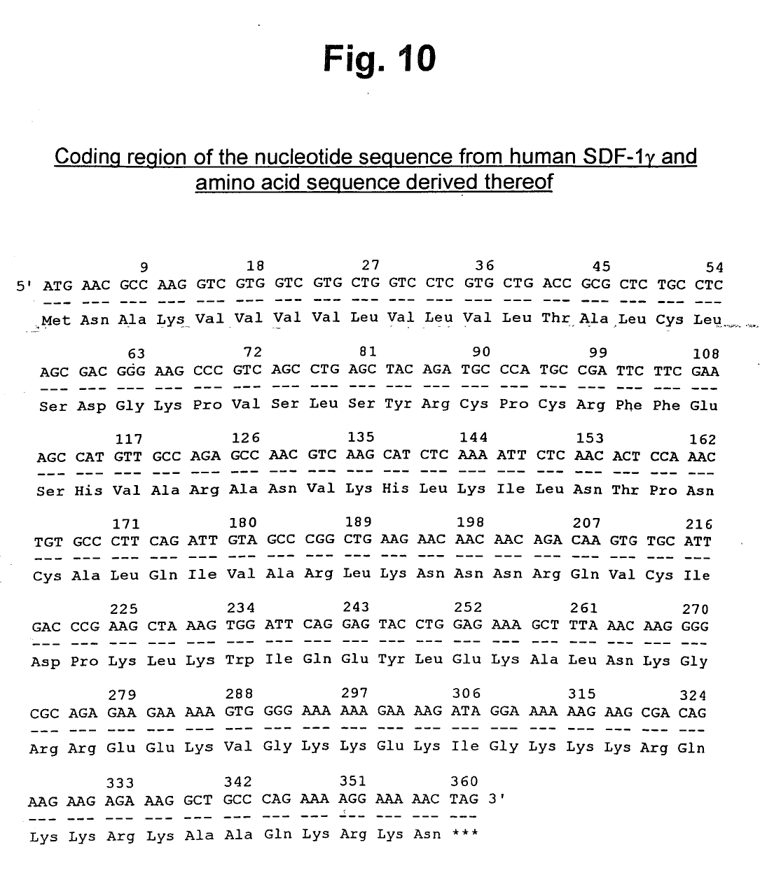

FIG. 10 shows the coding region of the nucleic acid sequence of human SDF-1γ and the amino acid sequence derived therefrom.

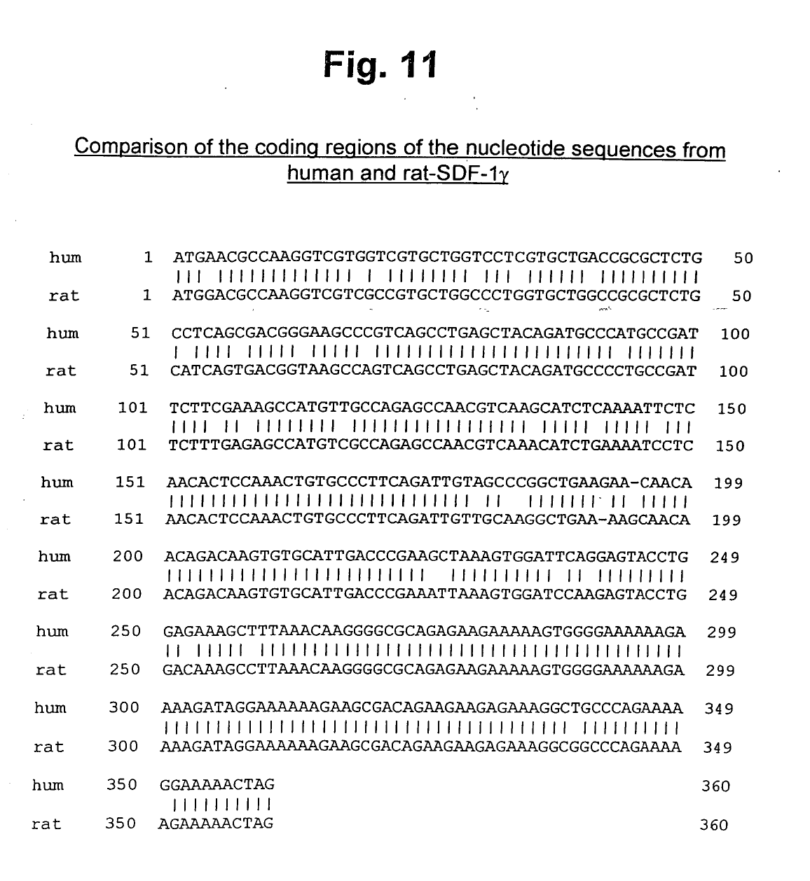

FIG. 11 shows a comparison of the coding regions of nucleic acid sequences of human and rat SDF-1γ. “hum”: human sequence; “rat”: rat.

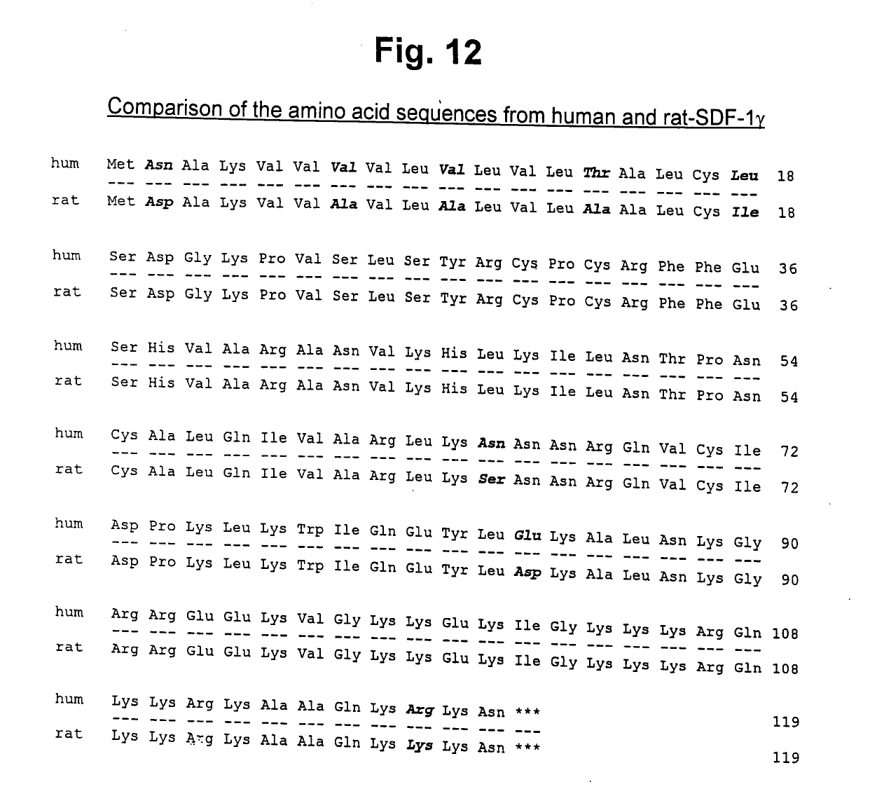

FIG. 12 shows a comparison of the amino-acid sequences of human and rat SDF-1γ derived from the nucleic acid sequences in FIG. 11. “hum”: human sequence: “rat”: rat.

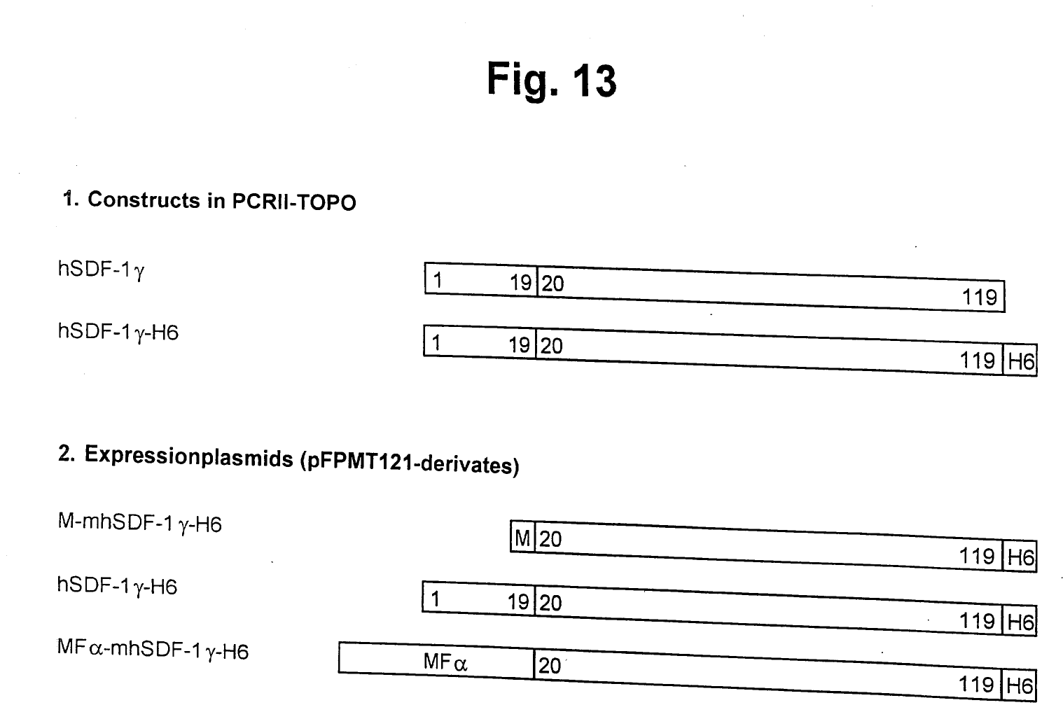

FIG. 13 shows schematically the hSDF-1γ and hSDF-1γ-H6 constructs in the plasmid PCRII-TOPO (Invitrogen, Groningen, NL) as well as the constructs M-mhSDF-1γ-H6, SDF-1γ-H6 and MFα-mhSDF-1γ-H6 in the plasmid pFPMT121.

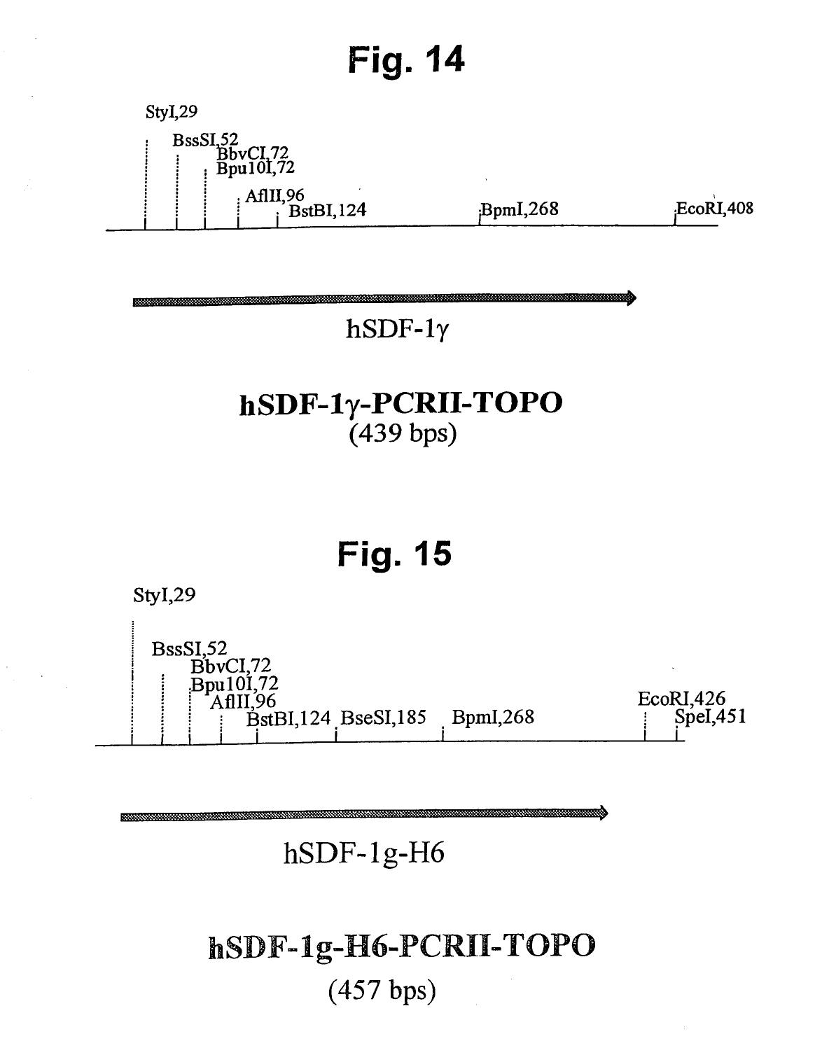

FIG. 14 shows the restriction map of the 439-bps-long DNA fragment with the coding region of the hSDF-1γ gene.

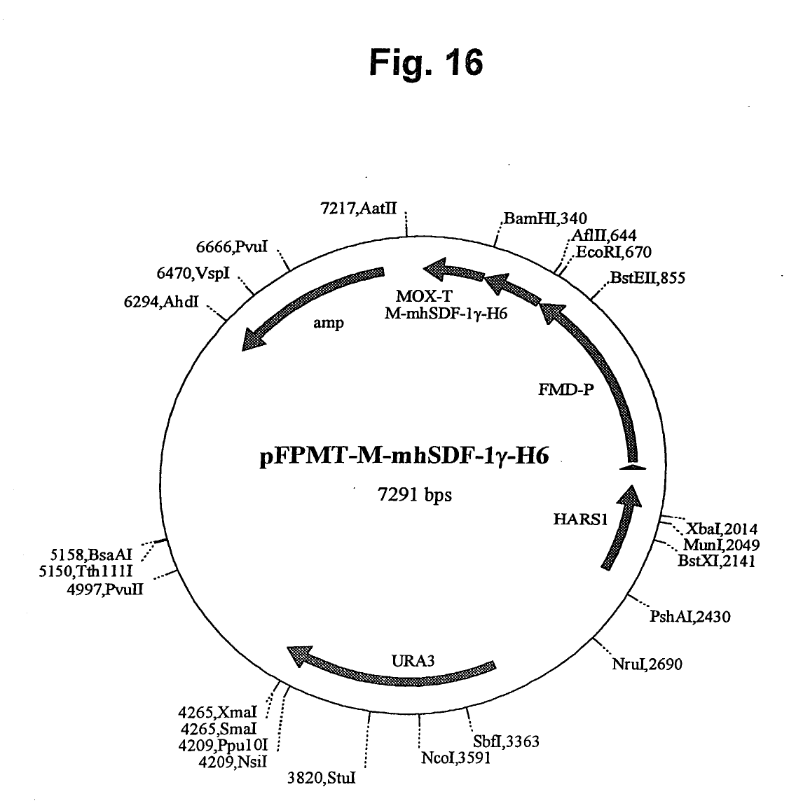

FIG. 15 shows the restriction map of the 457-bps-long DNA fragment with the coding region of the hSDF-1γ gene and the His tag.

FIG. 16 shows the restriction map of the expression plasmid pFPMT-M-mhSDF-1γ-H6.

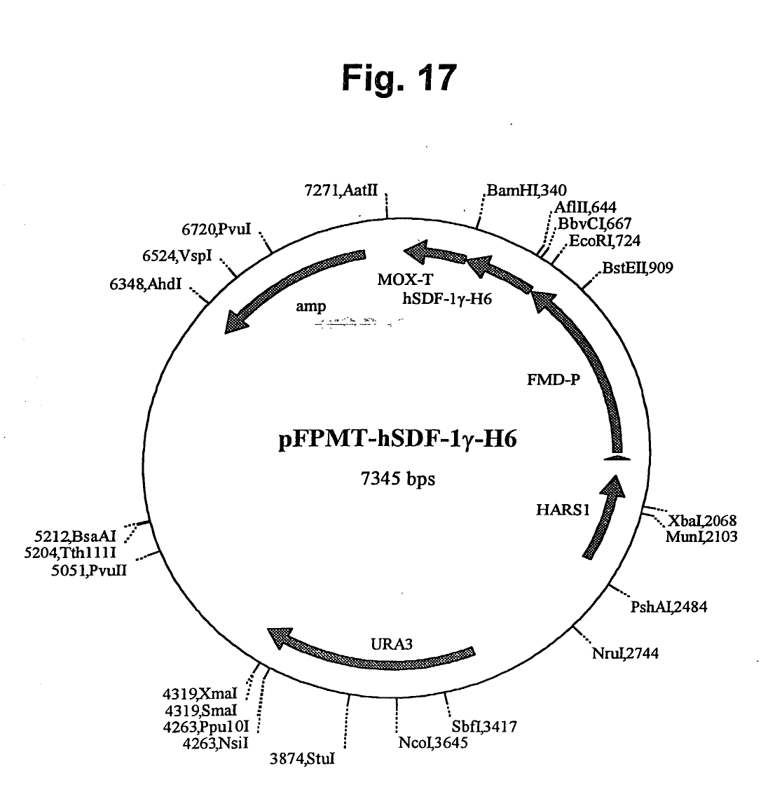

FIG. 17 shows the restriction map of the expression plasmid pFPMT-hSDF-1γ-H6.

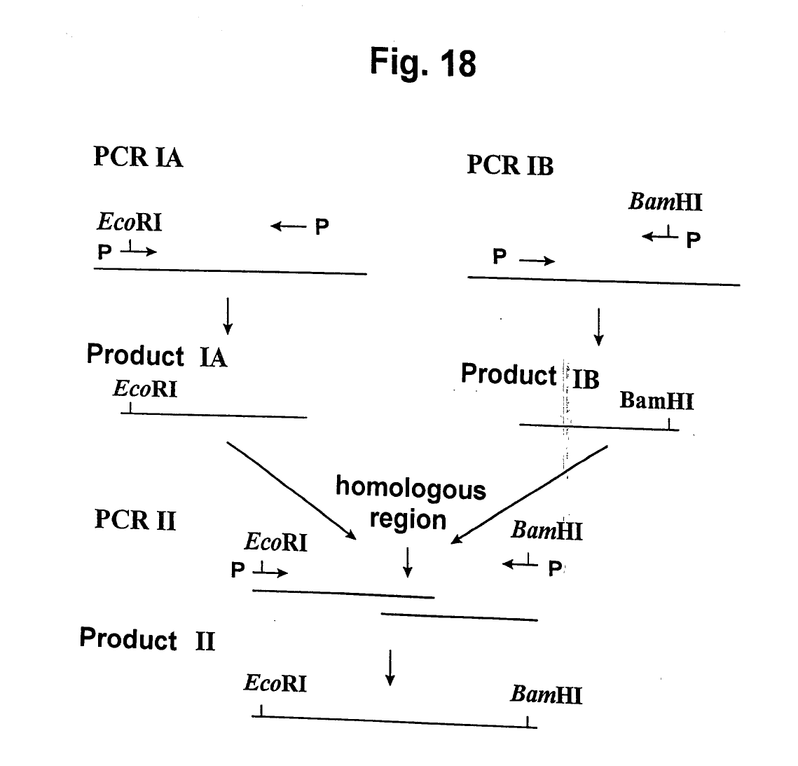

FIG. 18 shows schematically the strategy for the generation of expression plasmid pFPMT-MFα-mhSDF-1γ-H6. The arrows marked “P” represent PCR primer.

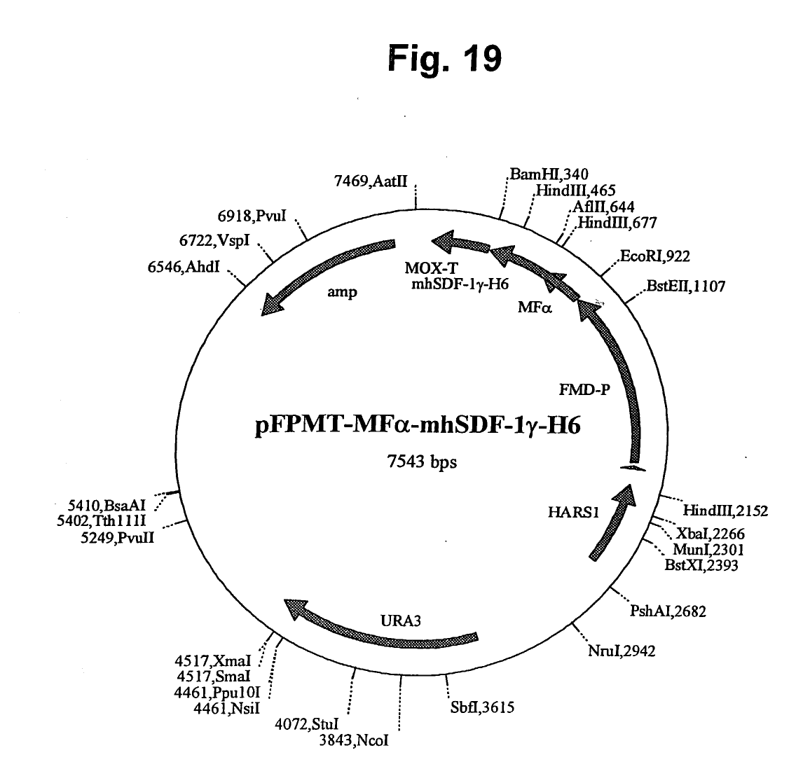

FIG. 19 shows the restriction map of the expression plasmid pFPMT-MFα-mhSDF-1γ-H6.

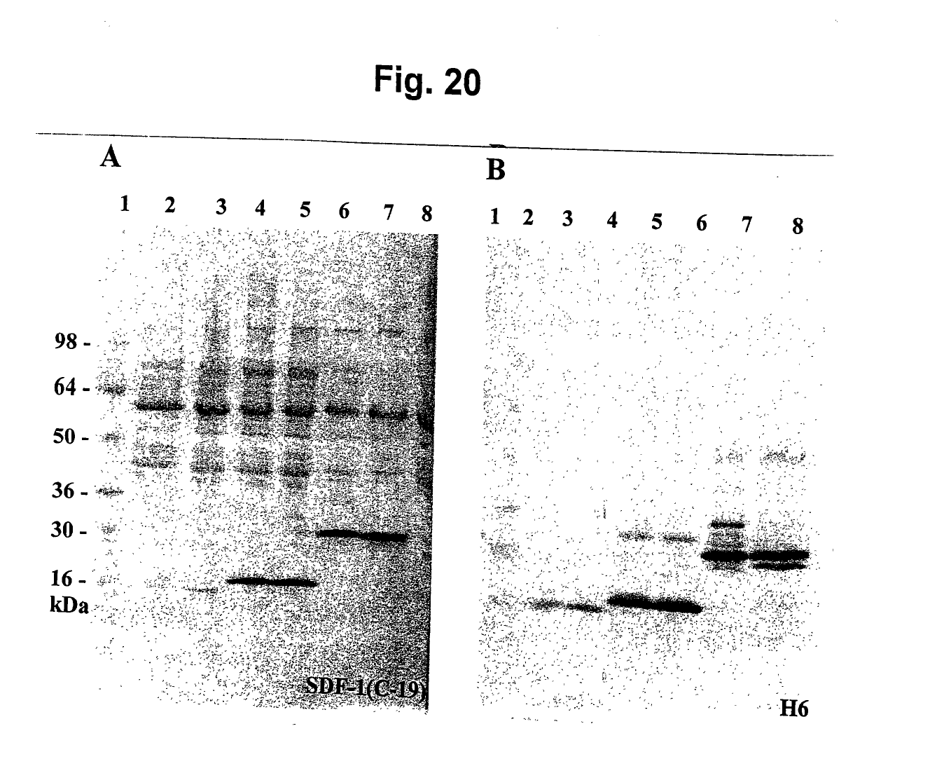

FIG. 20 shows the result of a Western Blot test for evidence of expression products in cell extracts of H. polymorpha (A) with the SDF-1-specific antibodies SDF-1(C19) (Santa Cruz Biotechnology, USA) and (B) with a His-tag-specific antibody (RGS-His Antibody, Mouse IgG1, Qiagen, Hilden, BRD). The tracks in (A) contain: (1) Sea Blue Prestained Standard, (2) M-mhSDF-1γ-H6, (3) M-mhSDF-1γy-H6 treated with PNGaseF, (4) hSDF-1γ-H6, (5) hSDF-1γ-H6 treated with PNGaseF, (6) MFα-mhSDF-1γ-H6, (7) MFα-mhSDF-1γ-H6 treated with PNGaseF and (8) cell extract without SDF-1γ. The tracks in (B) contain: (1) cell extract without SDF-1γ, (2) Sea Blue Prestained Standard, (3) M-mhSDF-1γ-H6, (4) M-mhSDF-1γ-H6 treated with PNGaseF, (5) hSDF-1γ-H6, (6) hSDF-1γ-H6 treated with PNGaseF, (7) MFα-mhSDF-1γ-H6 and (8) MFα-mhSDF-1γ-H6 treated with PNGaseF.

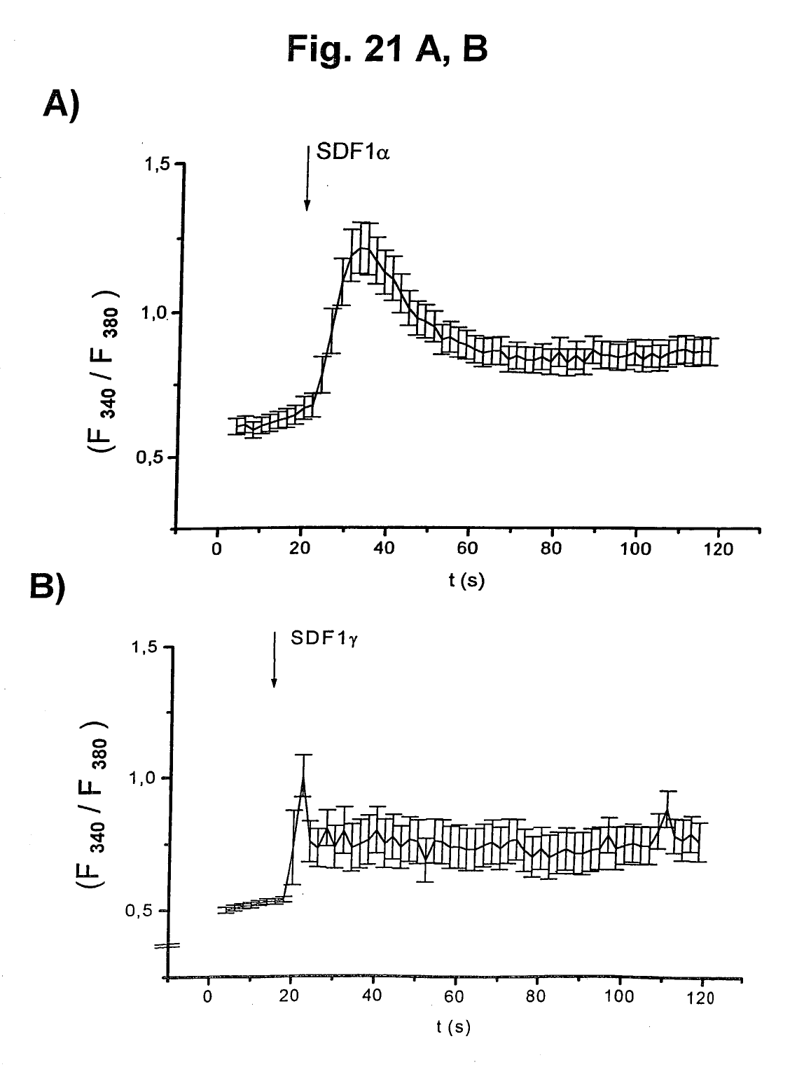

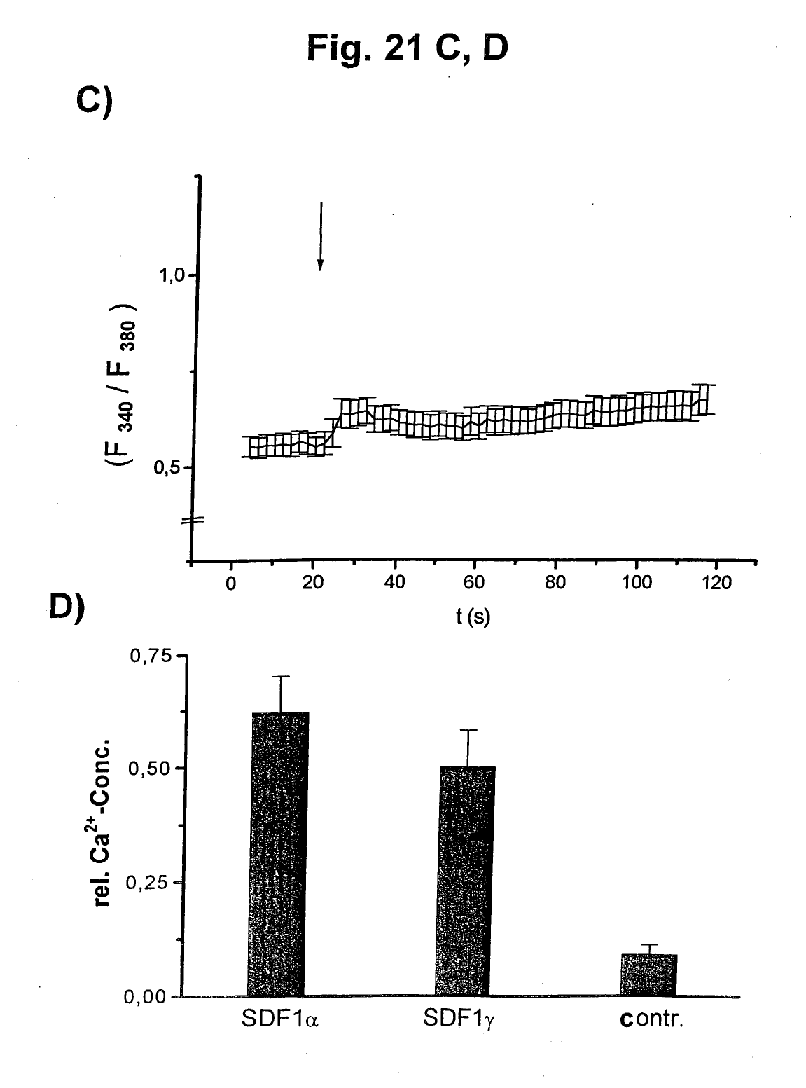

FIG. 21 shows a comparison of the effect of SDF-1α and SDF-1γ on the Ca concentration in astrocytes: (A) 50 nM SDF-1α; (B) 35 μg yeast cell extract with recombinant SDF-1γ (M-mhSDF-1γ-H6); (C) 22.4 μg control extract; (D) quantitative evaluation of the rise in intracellular calcium with SDF-1γ and the control extract in relation to the rise in calcium elicited by SDF-1α.

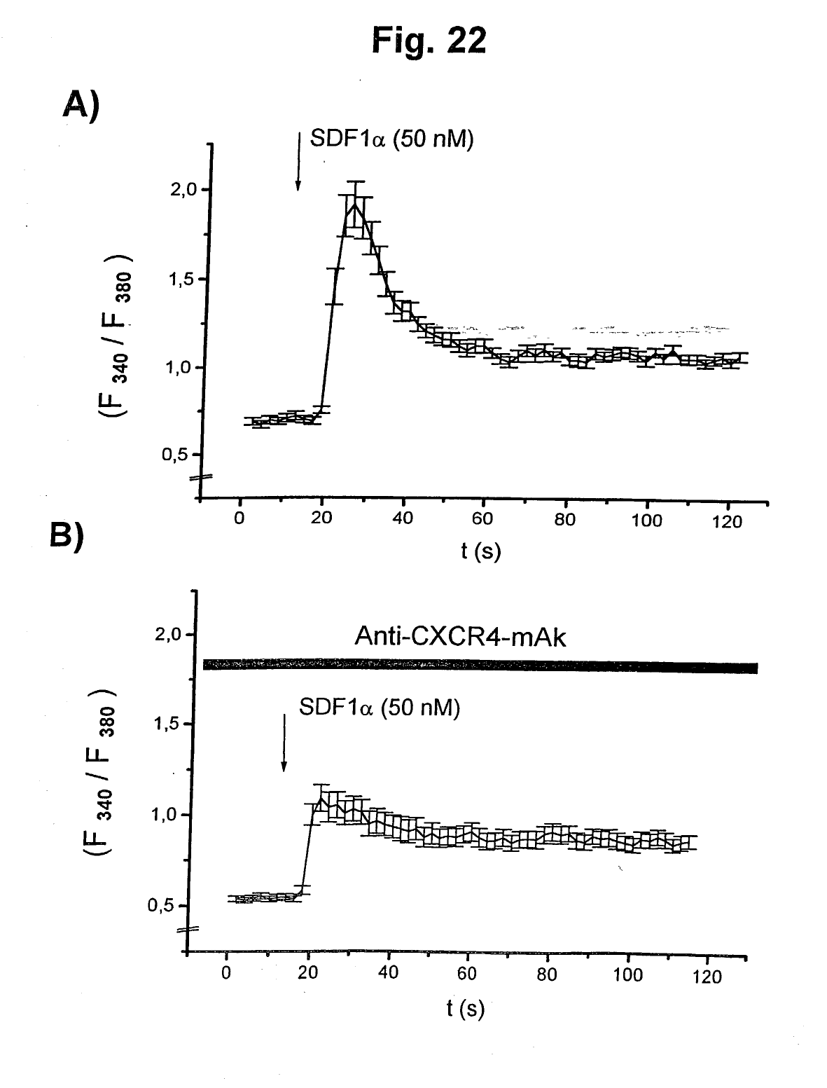

FIG. 22 shows the result of a Ca-imaging experiment in astrocytes for SDF-1α without (A) and with (B) pre-incubation with antibodies against CXCR4.

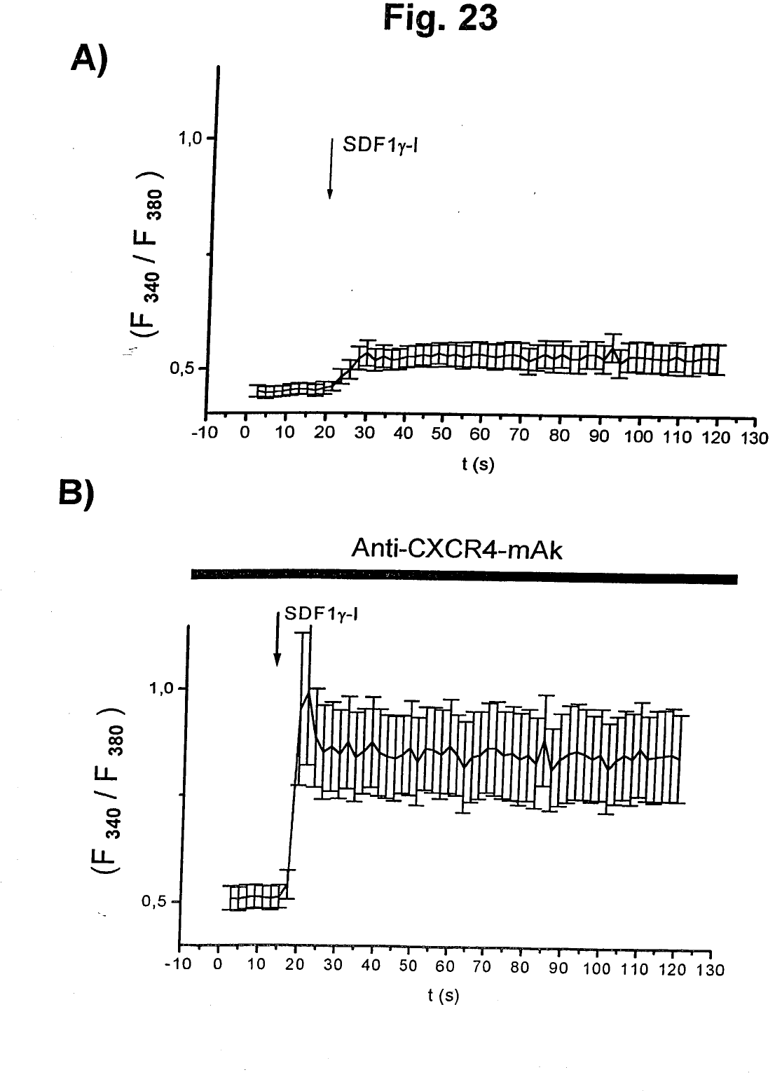

FIG. 23 shows the result of a Ca-imaging experiment in astrocytes for SDF-1γ without (A) and with (B) pre-incubation with antibodies against CXCR4.

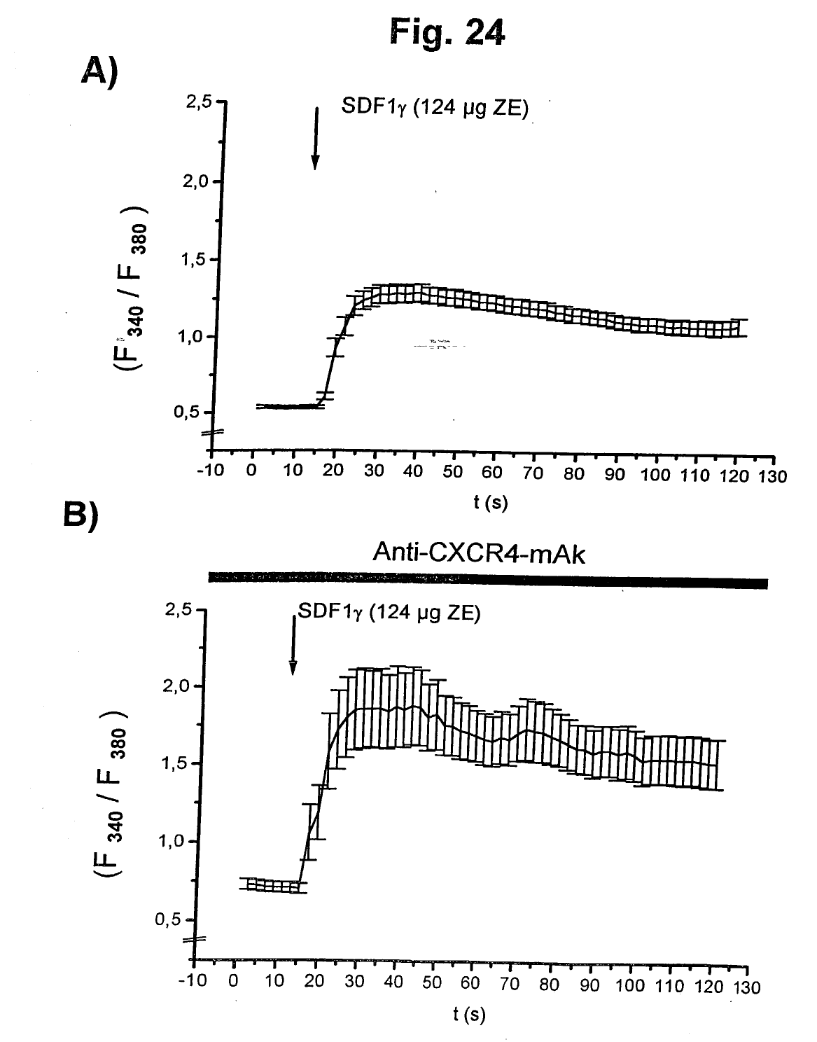

FIG. 24 shows the result of a Ca-imaging experiment in cortex neurones for SDF-1γ without (A) and with (B) pre-incubation with antibodies against CXCR4.

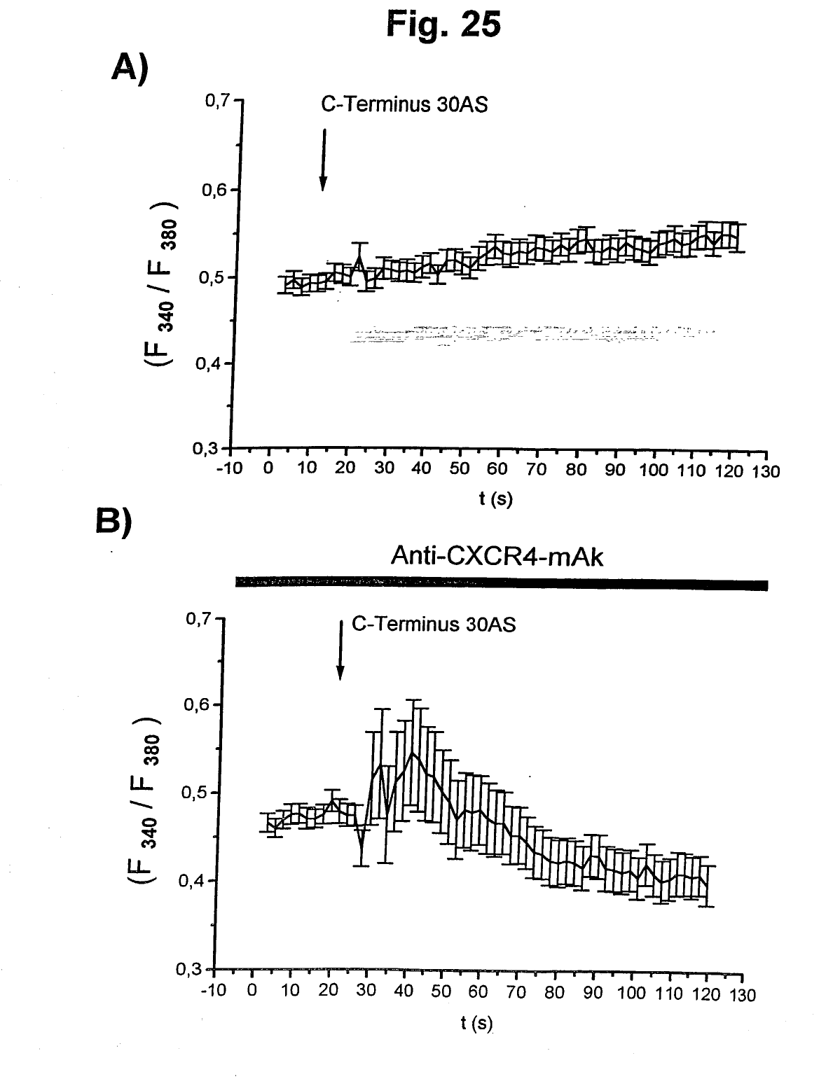

FIG. 25 shows the result of a Ca-imaging experiment in astrocytes for the C-terminal basic peptide of SDF-1γ (30 amino-acids) without (A) and with (B) pre-incubation with antibodies against CXCR4.

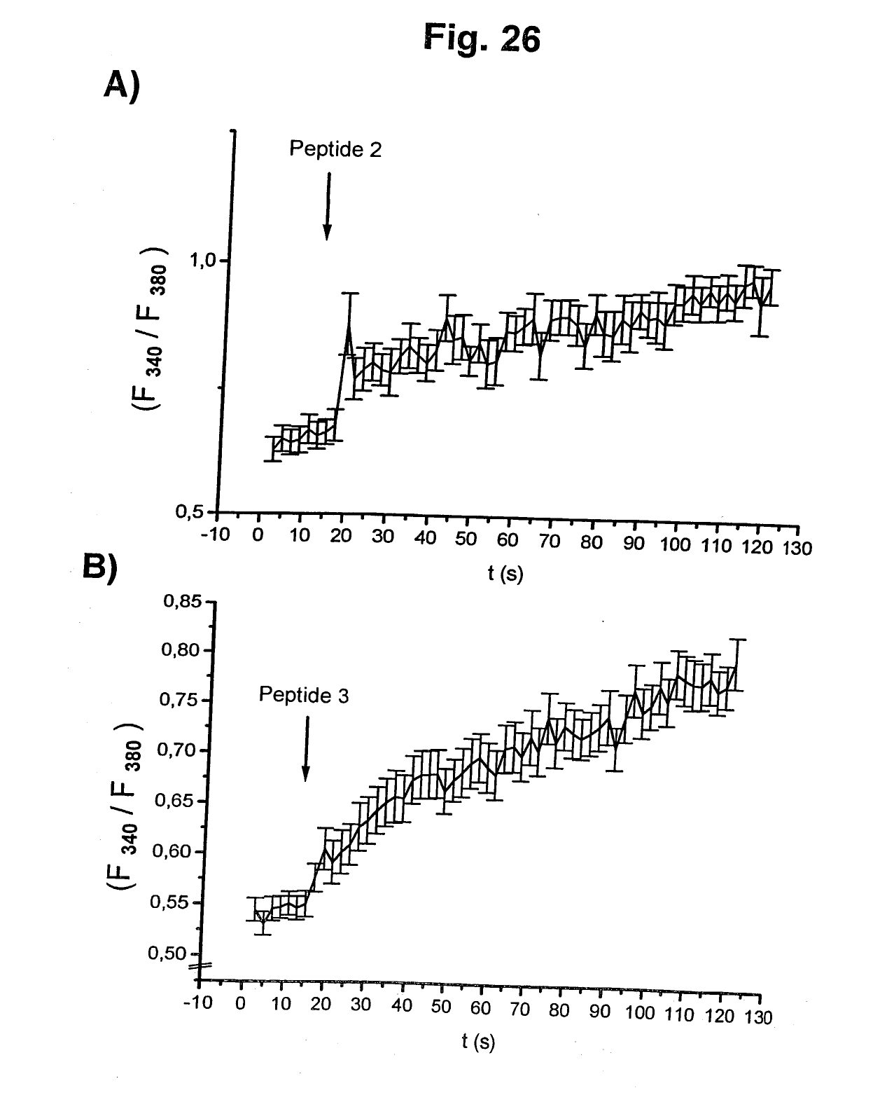

FIG. 26 shows the result of a Ca-imaging experiment in astrocytes for (A) peptide 2 (KKEKIG; SEQ ID NO:6) and (B) peptide 3 (KKKRQ; SEQ ID NO 8).

### EXAMPLES

### Example 1

### Cloning and Sequence Analysis of SDf-1γ

A. Materials and Methods

Animal experiments

Adult Wistar rats (body weight 200-250 g) were anaesthetized by the intraperitoneal administration of chloral hydrate (350 ml/kg body weight). The sciatic nerve in the upper thigh was compressed temporarily with pincers (Müiller et al., 1986). In order to obtain RNA from the nerve pathways the tissue 2-3 mm around the wound was removed and disposed of. All tests on animals were carried out in accordance with the guidelines of the German animal protection law.

Isolating RNA

Total RNA from rat tissues was isolated by the phenol-guanidine-thiocyanate process (Chomczynski and Sacchi, 1987. The frozen tissue specimens were homogenized twice for 45 seconds at 2500 rpm with a Polytron (Brinkmann, Westbury, USA). PolyA⁺ RNA was isolated by oligo(dT)-cellulose chromatography (Sambrook et al., 1988).

Construction of a cDNA Gene Bank

For the construction of a cDNA gene bank 4.5 μg Poly(A)⁺ RNA from the sciatic nerves of adult rats were used as template and oligo(dT)₁₂₋₁₈ as primer. cDNA was generated with the TimeSaver cDNA Synthesis Kit (Pharmacia LKB, Piscataway, N.J.). The cDNA was spliced by ligation by means of the Gigapack II packing extract (Stratagene) with γ-ZAP II phage particles previously resected with EcoRI. The titration of the cDNA gene bank obtained resulted in a complexity of about 0.5×10⁶. The screening of the gene bank was carried out by standard procedures (Sambrook et al., 1969) with a radioactively labelled cDNA fragment from the untranslated 3′ region of rSDF-1β (nucleotides 743-1368).

Oligonucleotides

The following oligonucleotides were synthesized with a GeneAssembler Plus Synthesator (Pharmacia, Piscataway, N.J.):

(SEQ ID NO:19)

MMSE2: 5′ ACGCCATGGACGCCAAGGTCG-3′
corresponds to the nucleotides 49-69
of rSDF-1β-cDNA.

(SEQ ID NO:20)

GAS2: 5′ -ACTGTAAGGAAGACCCTCTCTCACC-3′
corresponds to the nucleotides 2327-2303
of SDF-1γ.

(SEQ ID NO:21)

GAS3: 5′ -GTTGAGACTATGCATCGACTCCAAC-3′
corresponds to the nucleotides 2576-2552
of SDF-1γ.

DNA sequencing and analysis

Both cDNA strands of SDF-1β and of the 2.5 Kb-long insert in SDF-1γ including the banking regions were sequenced by the didesoxy-DNA sequencing method (Sanger et al., 1977) with the aid of T17 sequencing kits (Pharmacia-LKB). The sequences were confirmed by sequencing further independent clones from RT-PCR reactions. With the aid of the FASTA (Pearson 1980) and BLAST (Altschul et al., 1990) programmes the data were compared with the EMBL data bank. A more extensive analysis of the sequences was carried out with the aid of the PCGENE software package (Intelligenetics, Mountain View, Calif.).

RT PCR

The reverse transcription was carried out with 1.5 μg total RNA and Reverse-Transcriptase Superscript (Gibco, Gaithersburg) in accordance with the instructions of the manufacturers. The first cDNA strand was digested with RNase H (Boehringer Mannheim) and in addition 1/10 of the volume was used as a template for the PCR amplification with Amplitaq Polymerase (Pertkin Elmer) or Pfu Polymerase (Stratagene, La Jolla) (for SDF-1γ).

B. Cloning and Sequencing of SDF-1γ

In the identification of genes which following a nerve lesion are differentially expressed the cDNA clone NT-I-15 with 2174 nucleotides was isolated from a gene bank produced from the sciatic nerves of rats. The analysis of the sequence NT-I-15 clone indicated that this clone showed an 86% homology with the untranslated 3′ region (UTR) of mouse SDF-1β-cDNA (cf. Tashiro et al., 1993). On Northern Blot testing under strict washing conditions NT-I-15 hybridized with two transcripts from the sciatic nerve of adult rats (FIG. 2). While the smaller transcript corresponded to about 3 Kb of the size of SDF-1β mRNA, the longer transcript of 5.5 Kb was unknown. This transcript was named SDF-1γ.

The isolation of complete clones for both transcripts was tackled both by screening a gene bank and also by reverse transcription PCR (RT-PCR). Through screening of a gene bank from sciatic nerves of rats with a 626-nucleotide-long cDNA fragment of the NT-I-15 clone, corresponding to nucleotides 734-1368 of the 3′ UTR region of SDF-1γ of rats, a complete CDNA clone with a length of 2819 nucleotides was recovered, which contained the complete coding region of SDF-1γ.

Through renewed screening of the cDNA gene bank with the 626-nucleotide-long cDNA fragment of NT-I-15 an incomplete SDF-1 γ clone of about 3400 nucleotides was recovered which contained not only the complete 3′ UTR region and the last 4 codons (90-93) of SDF-1γ but also a new (non-coding) sequence with a length of about 1 Kb upstream of codon 90. It was then assumed that the transcript with a length of 5.5 Kb identified in the Northern Blot represented an alternatively spliced isoform which is produced by a 2.5-Kb-long insert between codons 89 and 90 of SDF-1β. In order to confirm this hypothesis a new fragment was produced through RT PCR with antisense primers which are specific for the new sequence at the 5′ end of the SDF-G6 clone (Primers GAS2 and GAS3) and a sense primer corresponding to the translation initiation site of SDF-1β (Primer MNSE2). The sequencing of the amplified PCR fragment indicated a transcript which downstream from codon 89 showed a sequence other than SDF-1β. This transcript coded for a peptide of 119 amino-acids, of which the first 89 amino-acids were identical with the first 89 amino-acids of SDF-1α and -β. Subsequent Northern Blot analyses confirmed that the sequence obtained represented the 5.5-Kb-long transcript. cDNA probes from the 3′ area common to SDF-1β and -γ (FIG. 2A) or from the 5′ region common to all the SDF isoforms of the entire 5′ area of the coding region (FIG. 2B) hybridized both with the 3-Kb-long (SDF-1β) and also with the 5.5-Kb-long (SDF-1γ) transcript, while a cDNA probe which was specific for the 2.5-Kb-long insert hybridized only with the 5.5 Kb SDF-1γ transcript (FIG. 2C). In the sciatic nerve of rats no SDF-1α-mRNA with a length of 1.5 Kb could be demonstrated.

Both strands of the SDF-1β cDNA of rats were sequenced. In SDF-1γ the new insert with a length of 2572 nucleotides and the flanking areas with the known SDF-1β were likewise doubly sequenced. The amino-acids derived for SDF-1β yield a peptide of 93 amino-acids with a theoretical molecular weight of 10.5 Kd. The first 19 amino-acids represent a signal peptide for proteins secreted. The amino-acid sequence derived for SDF-1β contains the first 89 amino-acid residues of SDF-1β and 30 additional amino-acids in the carboxy-terminal region which show no homology with SDF-1β (compare FIG. 3). The theoretical molecular weight of the 119-amino-acid-long SDF-1γ peptide is 13.5 Kd. The amino-acid sequence of the SDF-1β of rats shows a strong homology (96.8%) with the corresponding mouse protein (98.9% taking into account conservative amino-acid replacements). A comparison of the new SDF-1 isoforms SDF-1β and SDF-1γ with the known SDF-1 sequences is shown in FIG. 4.

### Example 2

### Demonstration of SDF-1β and SDF-1γ mRNA in Various Tissues and Developmental Stages

A. Northern Blot Analysis

Each 10 μg of total RNA was fractionated in 1.2% agarose gel containing 15% formaldehyde and then transferred by normal procedures to Nytran NY 13 N-membranes (Schleicher and Schull, Keene, N.H.). The filters were irradiated with UV light and dyed with methylene blue (Sambrook et al., 1989), prehybridized with 7% SDS in a 0.5M sodium phosphate solution and hybridized with 1-5×10⁶ cpm/ml of a ³²P-labelled cDNA probe in the same solution. cDNA fragments corresponding (i) to the whole 3′ UTR area of SDF-1β/γ (nucleotides 743-1368 in SDF-1β), (ii) to the total area coding for all the SDF-1 isoforms (nucleotides 49-239) and (iii) to a 1702-nucleotide-long segment of the SDF-1γ-specific insert (nucleotides 625-2357 in the SDF-1γ cDNA) were radioactively labelled by unidirectional PCR (Stürzl et al., 1991). After hybridization the filters were washed for at least 15 minutes at 60° C. in 2×SSC/1% SDS and for at least 15 minutes at 60° C. in 0.1×SSC/1% SDS. The filters were either exposed together with an X-ray film (X-Omat, Kodak) or quantified directly with a BAS 1050 Bioimager (Fuji).

B. Demonstration of SDF-1β and SDF-1γ mRNA in Various Tissues

The Northern Blot hybridization tests shown in FIG. 5A were carried out with total RNA from various tissues of adult rats and a 602-nucleotide-long fragment from the entire 3′ UTR region of SDF-1β/γ which had been radioactively labelled with ³²P-dCTP. The distribution of SDF-1β and SDF-1γ mRNA among various tissues showed a complementary pattern. While the β isoform was detected mainly in the liver, kidneys, spleen and thymus, SDF-1γ appeared predominantly in the tissues of the heart and lung as well as in mature tissues of the nervous system (FIG. 5). The fact that the SDF-1β transcript appears mainly above all in embryonic and neonatal brain tissues and in the sciatic nerve points to differential regulation of SDF-1 expression during the development of the nervous system. Neither SDF-1β nor SDF-1γ can be demonstrated in muscle and testicular tissues.

C. Demonstration of SDF-1β and SDF-1γ mRNA in the Brain and Sciatic Nerve in the Course of Development

Brain:

In the investigation of the development-specific distribution of SDF-1β and SDF-1γ RNA from the brain tissues at various stages of development in rats (from 17-day (E 17) embryos to adult rats) was tested with a 602-nucleotide-long fragment of the common 3′ UTR region of SDF-1β/γ. In the brain tissue of E17 embryos predominantly SDF-1β mRNA was demonstrated; the transcript amount diminished, however, with increasing age, and the transcript could no longer be demonstrated in the brain tissue of adult rats. By contrast, the amount of SDF-1γ transcript was very low in E17 embryos; it gradually increased and reached a maximum in adult rats (FIG. 5B).

Sciatic Nerve:

Total RNA was isolated from the sciatic nerve of rats at various stages of development (from 1 day following birth (P1) up to the attainment of the age of full growth). At P1 SDF-1β mRNA was demonstrated in small quantities; the transcript amount rose in the P4-P7 stage and fell below the demonstrable limit in the nerve tissue of adult rats (FIG. 5C).

SDF-1β and SDF-1γ mRNAs thus appear during development and in the nervous system of adult rats to show a different pattern. Whereas the SDF-1β isoform appears predominantly in the embryonic or perinatal central and peripheral nervous systems, SDF-1γ is the most important variant in the nervous system of adult rats.(FIG. 5B, C). In the period between the 4^{th} and 7^{th} days following birth, in which differentiation of glial cells and maturation of the neurones commences, the SDF-1β and SDF-1γ appear in nearly equal quantities.

D. Demonstration of SDF-1β and SDF-1γ-mRNA Following a Lesion of the Sciatic Nerve

Following injury to the sciatic nerve through compression, small alterations in the SDF-1β and SDF-1γ-mRNA patterns were observed at the distal end of the nerve. By means of “multiple quantitative imaging” of radioactive Northern Blot filters a temporary rise in the quantity of SDF-1β was determined, which reached a maximum of 175% two days after the nerve compression; following which the level fell until on the 7^{th} day after the compression it reached the same level as in the control. Testing established no significant change in the SDF-1γ mRNA following the nerve lesion.

### Example 3

### Cellular Localization of the SDF-1γ Transcript By in Situ Hybridization

A. In Situ Hybridisation

The tissue specimens were embedded in Tissue Tec II (Miles, Napperville, IL), frozen in methylbutane at −70° C. and cut into 20 μm thick sections. The sections were fixed and subsequently acetylated and prehybridized for 4 hours at 55° C. in accordance with Angerer et al. (1987). In vitro transcripts (i) of a sub-clone of the total 3′ UTR region of SDF-1β/γ (nucleotides 1758-2199 in SDF-1β), (ii) of a sub-clone of all the SDF-1 total isoforms of the total 5′ UTR and coding regions (nucleotides 1-596 in the SDF-1β cDNA) and (iii) of a sub-clone of the SDF-1γ-specific insert (nucleotides 661-1313 in the SDF-1γ cDNA) were produced with the aid of the DIG-RNA labelling kit of Boehringer Mannheim using digoxigenin UTP. Following hybridization at 55° C. overnight, a Rnase A-treatment (20 μg/ml in 0.6M NaCl, 20 mM tris HCI, 2 mM EDTA, pH8) was carried out for 20 minutes at 37° C. The sections were then washed three times with 2× SSC for 20 minutes each time at 50° C. and 3 times with 0.2× SSC for 20 minutes each time at 50° C. The demonstration of digoxigenin was carried out according to the instructions of the manufacturer (Boehringer Mannheim).

B. Cellular Localization of the SDF-1γ Transcript

For the in situ hybridization, antisense transcripts from (a) the common 3′ UTR region of SDF-1β/γ, (b) the coding and 5′ UTR region common to all the SDF-1 isoforms and (c) the SDF-1γ-specific insert labelled with digoxigenin-UTP. The sense transcripts serve as negative controls.

In the CNS of adult rats strong and pronounced hybridization signals were observed in regions with cerebral grey matter both in “pearl-necklace”-like rows of oligodendrocytes in myelinized nerve phases as well as for instance in the corpus callosum (FIG. 5A, B). Further hybridization signals appear particularly in association with Purkinje and granular neurones in the cerebellum (FIG. 6D, E), in pyramidal and granular neurones in the hippocampus (FIG. 6G, H) as well as in neurones of all the main layers of the neocortex (FIG. 7A, B). No hybridization signals are obtained with the corresponding sense transcripts (see FIG. 6C, F, I for the sense probes for SDF-1γ).

The signals obtained with the SDF-1γ-specific antisense transcript were almost identical with the hybridization pattern obtained with the entire SDF-1 antisense probe, which thereby indicates that the SDF-1γ isoform is expressed in neurones and oligodendrocytes of the brain of adult rats. SDF-1β transcripts, insofar as they occur in the brain of adult rats, appear to be present in the same regions and cell populations as SDF-1γ.

Longitudinal section of the sciatic nerve of adult rats evokes from the entire 3′ region an antisense probe of SDF-1β/γ spindle-shaped hybridization signals which suggest the typical shape of Schwann cells close to axon phases (FIG. 8A, B). The identical localization Of S-100 immunoreactivity as well as of the hybridization signals in transverse sections of the sciatic nerve (see arrow-points) confirmed the expression of SDF-1β/γ in Schwann cells (FIG. 8 D, E). Using an SDF-1γ-specific antisense probe one obtains a labelling pattern in the sciatic nerve of adult rats (FIG. 8H) which strongly suggests the hybridization pattern of the antisense transcripts common to all the SDF isoforms (FIG. 8G). SDF-1γ RNA (and presumably also the SDF-1β isoform) occurs both in Schwann cells (see arrow-points) and in vascular cells of the sciatic nerve (see upper right corner in FIG. 8G, H). The results of the in situ hybridization tests agree with the results of the Northern Blot tests of FIGS. 2 and 3. SDF-1α mRHA was not demonstrated with antisense/sense transcripts from the SDF-1α-specific 3′ region either in the brain or in the sciatic nerve of adult rats.

### Example 4

### Expression of SDF-1γ products in Hansenula polymorha

A. hSDF-1γ Constructs

For the expression of SDF-1γ in Hansenula polymorpha three different constructs were produced, with the objective of providing better analytical approximation to a His-tag. The constructs are shown in FIG. 13.

1. M-mhSDF-1γ-H6 (methionine/mature human SDF-1γ/His-tag. In this fusion protein the sequence of mature human SDF-1γ (amino-acids 20-119 in SEQ ID NO:12) is located at the end of an N-terminal methionine residue. Since no leader sequence is present, a cytosolic localization was expected. At the C-terminus six histidine residues (His-tag) are located.

2. hSDF-1γ-H6 (immature human SDF-1γ/His-tag). This construct contains amino-acids 1-119 of SDF-1γ (SEQ ID NO:12) followed by a C-terminal His-tag. It consequently comprises the natural leader sequence known in human cells. Since leader sequences are occasionally also recognized in heterologous host cells, whether H. polymorpha recognizes the authentic SDF-1γ leader peptide should be investigated with this construct.

3. MFα-mhSDF-1γ-H6 (pre-pro-sequence of the mating factor α from Saccharomyces cerevisiae mature human SDF-1γ/His-tag). The sequence of mature SDF-1γ (amino-acids 20-119 in SEQ ID NO:12) at the end of the pre-pro-sequence of mating factor a from the related brewing yeast Saccharomyces cerevisiae often used in H. polymorpha. This construct ought to be secreted by H. polymorpha.

B. Construction of Expression Plasmids

The plasmid SDF-1γ-PCRII-TOPO, which contains the 439-bps-long SDF-1γ insert (FIG. 14), served as the basic construct. In a first step six codons for a C-terminal His-tag were enclosed in the hSDF-1γ sequence (hSDF-1γ-H6, FIG. 15).

As basic vector for the later expression of SDF-1γ constructs in H. polymorpha the integrative plasmid pFPMT121 (Gellissen, 2000), in which the foreign gene to be expressed stands under the control of the FMD promotor, was inserted. On the foundation of this plasmid the following expression vectors were constructed:

1. pFPMT-M-mh SDF-1γ-H6. By means of PCR a DNA fragment in which the coding sequence of SDF-1γ is flanked by an EcoRI- (ahead of the starting codon) and a BamII restriction excision site, was generated. hSDF-1γ-H6 in PCRII-TOPO (Invitrogen, Groningen, NL) served as template. The PCR product was digested with EcoRI/BamHI and cloned between the corresponding sites of the pFPMT121 plasmid. The map of the resulting plasmid pFPMT-M-h SDF-1γ-H6 is shown in FIG. 16.

2. pFPMT-hSDF-1γ-H-6. In this construct, too, a PCR product flanked by an EcoRI and a BamHI resection site was first constructed, in which hSDF-1γ-H6 in PCRII-TOPO once again served as template DNA. The PCR product digested with EcoRI and BamHI was cloned between the corresponding sites of the pFPMT121 plasmid. The map of the resulting plasmid pFPMT-hSDF-1γ-H6 is shown in FIG. 17.

3. pFPMT-MFα-mhSDF-1γ-H6. For the generation of this plasmid two separate PCR products were constructed (see FIG. 18). The first PCR product (PCR1A) contained the codons of the prepro-sequence of the mating factor α, flanked by an EcoRI resection site (before the starting codon) and at the other end by bases with homology to the first codons of the mature hSDF-1γ sequence. The second PCR product (PCR1B) contains the sequence of mature hSDF-1γ, in the foremost part flanked by bases with homology to the hindmost part of the prepro-sequence of mating factor a, in the hindmost part flanked by a Bam-II resection site (at the end of the stop-codon). Then a further PCR reaction was performed, in which the products of both the PCR1A and PCR1B described above were mixed. As primer the forward primer from PCR1A (that with the EcoRI resection site) and the backward primer from PCR1B (that with the BamII resection site) were inserted. The resultant PCR product contained the prepro-sequence of mating factor a fused with the sequence of mhSDF-1γ, flanked by EcoRI (before the starting codon) and BamHI (after the stop codon). After digestion with EcoRI BamHI the fragment was cloned between the corresponding resection sites of the pFPMT121 plasmid. The map of the resulting plasmid pFPMT-MFα-mhSDF-1γ-H6 is shown in FIG. 19.

C. Transformation of H. polymorpha with the Expression Vectors Produced.

Generation and Identification of Strains.

For the manufacture of competent H. polymorpha cells for electroporation 5ml YPD medium was inoculated with a single colony of H. polymorpha RB11 (odc, orotidin-5-phosphate-decarboxylase-deficient (uracil-auxotrophic) H. polymorpha strain (Weydemann et al., 1995) and shaken for 16 hours at 37° C. Subsequently 100 ml YPD medium in a 2 l. Erlenmeyer flask was inoculated with 3 ml of this preliminary culture and incubated at 37° C. to an OD₅₀₀ of 0.8-1 (vibration frequency 140 rpm). The cell harvest followed, through centrifugation of the culture in 50 ml Falcon tubes (4000 rpm, 6′) in a Beckmann centrifuge. After removal of the supernatant the cells were resuspended in 20 ml 50 mM potassium phosphate buffer (pH 7.5, prewarmed to 37° C.), mixed with 0.5 ml 1M DTT and incubated (waterbath) at 37° C. for 15′.

After this the cells were again centrifuged down (3000 rpm; 10′; Beckmann centrifuge), washed in 100 ml, then 50 ml, STM buffer (270 mM saccharose; 10 mM tris-HCl pH 7.5, 1 mM MgCl₂). After further centrifugation the cells were resuspended in 0.5 ml STM buffer and as 60 μl aliquots either used directly for transformation or frozen at −70° C. for use later.

Competent cells of H. polymorpha were transformed as follows with the three expression plasmids constructed (see above): 60 μl of competent H. polymorpha were mixed with 1-2 μg of the introductory circular plasmid DNA and transferred to electroporation cuvettes with 2 mm wide apertures. Electroporation followed at 2 kV, 25 μF and 200 ohms. Subsequently the cells were transferred to test-tubes with 1 ml YPD medium and agitated for one hour at 37° C. (angle 45°, 160 rpm). Following this recovery each 330 μl of cells was plated out on YNB-agar plates (1% glucose; without uracil). The plates were incubated at 37° C. until macroscopic uracil-prototrophic colonies were visible (about 1 week).

Thereupon, each of 36 uracil-prototrophic colonies were converted to stable strains through fourfold passaging and twofold stabilization. For passaging each 2 ml of YNB medium (1% glucose) was inoculated with single uracil-prototrophic colonies from the transformant plates and incubated for 2 days at 37° C. (angle 45°, agitation frequency 160 rpm). Each 150 μl of the resulting cultures was transferred to 2 ml fresh YNB medium and once again incubated for 2 days (see above). This operation was carried out four times (=four passages). For stabilization each 150 μg of the cultures from the latest passage was transferred into 2 ml YPD medium and incubated for 2 days at 37° C. (see above). Subsequently aliquots of these cultures were plated out on YNB-agar plates (without uracil). One single colony per cultivation was isolated and defined as a strain.

D. Induction of Expression and Demonstration of SDF-1γ Products

After isolation all strains were subjected to an MeOH induction and the soluble intracellular fractions analysed by Western Blotting regarding their content of hSDF-1γ products. First of all, each 2ml YPD medium in a 10 ml test-tube was inoculated with single colonies of the strain to be tested, and then for induction of expression of the foreign gene incubated at 37° C. for 16 hours (angle 45°, agitation frequency 160 rpm). Subsequently 150 μl of the resultant thick growth of cultures was placed as inoculum each in 3 ml YNB medium (1% glycerine). After 24 hours' agitation at 37° C. the cells were centrifuged down and each resuspended in 3 ml YNB medium (1% MeOH). Expression of the foreign gene was then induced by agitating again for 24 hours at 37° C.

After centrifugation of the cells from the induction cultures aliquots of the supernatant were mixed with 4×SAB (8% w/v SDS; 40% w/v glycerine; 8 mM EDTA pH 6.8; 250 mM tris pH 6.8; 0.4% w/v bromphenol blue; 40% v/v α-mercaptoethanol) and denatured for 5′ at 95° C. for the preparation of culture supernatants.

For preparation of intracellular soluble fractions the following steps are carried out on ice or at 4° C. The cell pellets from the induction cultures are resuspended each in 500 μl extraction buffer (50 mM tris pH 7.5, 150 mMNaCl, 0.1% v/v Triton X100 or PBS buffer) and each mixed with 12.5 μl PMSF. The specimens are subsequently transferred to 1.5 ml Eppendorf vessels. After addition of 500 μl glass beads cell disruption followed in a Vibrax at 2500 rpm. The supernatant was transferred to fresh Eppendorf vessels and centrifuged for 10′ at 10,000 rpm (Eppendorf centrifuge with cooling function). The supernatants of this centrifugation represented the so-called soluble intracellular fraction. For direct protein gel electrophoresis these were mixed with ¼ vol. 4×SAB and denatured for 5′ at 95° C., or frozen without addition of SAB at −20° C. for later use.

For the PNGaseF digestion each 8 μl of native intracellular soluble fraction was mixed with 1 μl 1% SDS and incubated for 5′ at 95° C. Then there followed addition of 1 μl PNGaseF (2 μ, Roche) or H₂O. Following incubation at 37° C. for 16 hours 4 μl 4×SAB were added, the specimens denatured for 5′ at 95° C. and separated on protein gels.

The separation of the denatured specimens by protein gel electrophoresis followed on 4-20% tricine-SDS gels (Novex) according to the manufacturer's directions. Subsequently the protein bands were transferred to nitrocellulose membranes in a Semi-Dry-Blot apparatus (Trans-Blot SD; Biorad) according to manufacturer's directions. For the Western Blots a His-tag-specific monoclonal antibody from the mouse (RGS-His-Antikorper, Qiagen, Hilden, BRD) or an SDF-1-specific polyclonal serum from the goat (SDF-1 (C19); #sc6193; Santa Cruz Biotechnology, USA) were used as primary antibodies (sera). The Western Blots were performed with the Western Breeze Kits Mouse or Goat (Novex) in accordance with manufacturer's instructions.

In this way, strains which produced significant amounts of the particular hSDF-1γ-H6 derivatives could be identified for each of the three constructs. For further product analyses in each case the most productive strain was chosen. For pFPMT-M-mhSDF-1γ-H6 this was the g7-5/36 strain; for pFMPT-hSDF-1γ-H6 and pFMPT-MFα-mhSDF-1γ-H6 the strains g8-28/7 and g9c-20/6 were correspondingly selected.

E. Product Analyses

In the culture supernatants of strains g7-5/36, g8-28/7 and g9c-20/6 no secreted SDF-1γ products could be detected by means of Western Blot. In the intracellular soluble fraction of these strains SDF-1γ products could be identified both with a His-tag specific antibody from the mouse and with an SDF-1γ-specific serum from the goat (SDF-1 (C19); #sc6193; Santa Cruz Biotechnology, U.S.A.) (see FIG. 20 A, B). The intracellular soluble fraction of a control strain (without SDF-1γ) did not show the products identified as SDF-1γ products (FIG. 20, track 8 (A), track 1 (B)).

The molecular weights of the main SDF-1γ products observed on Western Blot generally lie somewhat above the calculated molecular weights. M-mhSDF-1γ-H6: 12,692 kDa calculated, about 16 kDa observed (FIG. 20, tracks 2 and 3 (A); tracks 3 and 4(B)); hSDF-1γ-H6: 14,529 kDa calculated, about 17 kDa observed (FIG. 20, tracks 4 and 5 (A), tracks 5 and 6 (B)); MFα-mhSDF-1γ-H6: 21,468 kDa calculated, about 30 kDa observed (FIG. 20, tracks 6 and 7 (A), tracks 7 and 8 (B)). Since all the main bands are detectable both with the His-tag-specific antibodies and with the SDF-1-specific serum, the proteins belonging to the bands must be integral to the C-terminal. Furthermore the apparent molecular weights of the various products show the anticipated relative gradations M-mhSDF-1γ-H6<hSDF-1γ-H6<MF-α-mhSDF-1γ-H6, FIG. 20).

The amino-acid sequences of M-mhSDF-1γ-H6 and hSDF-1γ-H6 include no potential N-glycosylation sites. Correspondingly, PNGaseF digestion has no influence on the apparent molecular weight of particular main product bands (FIG. 20, tracks 2/3 and 4/5 (A), tracks 3/4 and 5/6 (B)). MFα-mhSDF-1γ-H6 has three N-glycosylation sites in the area of the MFα pre-pro sequence, which typically become N-glycosylated in the ER*. The absence of reduction of the apparent molecular weight of the 30 kDa product following PNGaseF digestion indicates that in this product what is concerned is the pre-pro product which is not incorporated into the ER (FIG. 20, tracks 6/7 (A), tracks 7/8 (B). Above 30kDa there are three weak PNGaseF-sensitive bands (track 7 (B)), which following N-deglycosylation are shifted to below 30 kDa (track 8 (B)). These bands can be interpreted as N-glycosylated pro-forms of MFα-mhSDF-1γ-H6 on the pro-sequence, from which the pre-sequence is split off during entry into the ER.

### Example 5

Effect of Recombinant Human SDF-1γ on the Calcium Concentration in Nerve Cells and Glial Cells

In this example the effect of recombinant human SDF-1γ (M-mhSDF-1γ-H6} in cell extracts of Hansenula polymorpha on the calcium concentration in nerve cells and glial cells is investigated. For estimation of the calcium concentration the calcium-imaging method was used (see Köller et al., 2001).

For the calcium imaging experiments primary astrocytes and primary cortex neurones of rats (Wistar strain) from newborn rats (post-natal day 0-1; astrocytes) or from rat embryos (embryonal day 15; cortex neurones) were prepared and cultivated as described by Köller et al. (2001). After 5-15 days in culture the cells were loaded for 1 hour in vitro with the calcium indicator Fura-2. The intracellular Fura-2 reacts with liberated calcium to form Fura-calcium, which has a different absorption wavelength (340 nm) from Fura-2 (380 nm). With the aid of the extinction coefficient (F340/F380) the relative intracellular calcium can be determined and graphically delineated. This procedure enables the detection of changes in the intracellular calcium concentration which are elicited by extracellular stimuli (e.g. ligand/receptor interactions).

FIG. 21 shows the result of Ca-imaging experiments, in which the effects of SDF-1α and SDF-1γ on the Ca concentration in astrocytes are compared. After the application of SDF-1α (50 nM, R&D Systems, Wiesbaden, BDR) the calcium concentration in cultivated astrocytes rises (FIG. 21 A). Following application of yeast cell extract with recombinant SDF-1γ (M-mhSDF-1γ-H6) (36 pg total protein) a rise in intracellular calcium results in cultivated astrocytes (FIG. 21 B). The response, however, is somewhat more limited than the response to SDF-1α.

Following application of control extract (22.4 μg protein from a cell extract of H. polymorpha RB11 cells which were transformed with the pFPMT121 plasmid without insert) the rise in intracellular calcium in cultivated astrocytes is absent (FIG. 21 C).

FIG. 21 D shows the quantitative assessment of the rise in intracellular calcium with SDF-1γ and the control extract related to the rise in calcium elicited by SDF-1α.

In addition it was tested whether the pre-incubation of the cells with a CXCR4-specific antibody (monoclonal antibody 12G5; R&D Systems, Wiesbaden, BRD) can reduce such a calcium response, as was previously observed for the SDF-1α-induced calcium reaction (for the detailed methodology in calcium-imaging experiments see Köller et al., 2001).

FIG. 22 shows the result of a Ca-imaging experiment in astrocytes for SDF-1α (A) without and (B) with antibody against CXCR4. FIG. 23 shows the result of the corresponding experiments for SDF-1γ. Following application of SDF-1α (50 nM, R&D Systems, Wiesbaden, BRD) the intracellilar calcium concentration in cultivated astrocytes rises sharply (FIG. 22 A). If, however, one gives SDF-1α after 5 minutes' pre-incubation with monoclonal antibody 12G5, cultivated astrocytes show an intracellular calcium outflow reduced by about 50% (FIG. 22 B). These results confirm findings in the literature, that the influence of SDF-1α or -1γ on the intracellular calcium concentration in astrocytes from the central nervous system is mediated through the CXCR4 receptor.

Following application of 35 μg yeast cell extract with recombinant SDF-1γ (M-mhSDF-1γ-H6; cell contents in PBS buffer) a measurable calcium increase results in cultivated astrocytes (FIG. 23 A). Unlike with SDF-1α the application of SDF-1γ to cultivated astrocytes which have been pre-incubated for 5 minutes with the monoclonal antibody 12G5 against the CXCR4 receptor leads to a sharply increased significant intracellular calcium discharge. (FIG. 23 B).

Following pre-incubation with the CXCR4 antibody cell cultures of cortex neurones from the rat brain also show a further increase in calcium concentration as a reaction to SDF-1γ. FIG. 24 shows the result of a corresponding Ca-imaging experiment in cortex neurones. The application both of 35 μg and of 125 μg (total protein) of a yeast extract with recombinant SDF-1γ (M-mhSDF-1γ-H6) leads to a significant increase in the calcium concentration in cultivated primary cortex neurones (FIG. 24 A). After 5 minutes' pre-incubation with the monoclonal antibody 12G5 (antibody against CXCR4) the addition of SDF-1γ occasions a sharply increased intracellular calcium discharge in cultivated primary cortex neurones (FIG. 24 B).

These above results confirm that the cell physiological reaction of neurones and astrocytes from the central nervous system to SDF-1α and/or SDF-1β clearly differ from the reactions to the new chemokine SDF-1γ.

### Example 6

Effect of the C-terminal Basic Peptide from SDF-1γ and the Synthetically Manufactured Peptide Breakdown Products Derived Therefrom on the Intracellular Ca-Concentration in Astrocytes

It was first investigated how the addition of a basic peptide with an amino-acid sequence corresponding to the last 30 amino-acids in the C-terminal region of SDF-1γ affected the-intracellular Ca concentration in astrocytes.

FIG. 25 shows the result of a Ca-imaging experiment with the C-terminal basic peptide of SDF-1γ in astrocytes. The application of 1 μg/ml of the synthetic peptide representing the C-terminal 30 amino-acids of SDF-1γ exerts only a weak influence on the intracellular Ca concentration of cultivated astrocytes (FIG. 25 A). If the astrocytes have previously been incubated for 5 minutes with the monoclonal antibody 12G5 (antibody against CXCR4), a sharply increased intracellular discharge of calcium into the primary astrocytes results on application of the same C-terminal peptide (FIG. 25 B).

In a Ca-imaging experiment with the peptides RREEKVG (Peptide 1, SEQ ID NO:5), KKEKIG (Peptide 2, SEQ ID NO:6), KKKRQ (Peptide 3, SEQ ID NO:8), KKRKAAQ (Peptide 4, SEQ ID NO:9) and KKKN (Peptide 5, SEQ ID NO:10) as well as with the amidized Peptides 1′ (RREEKV(NH2)) and 2′ (KKEKI(NH2)) it was established that the addition of the unamidized Peptides 1, 2 and 3 as well as the amidized Peptides 1′ and 2′, leads to an increase in the intracellular Ca concentration in astrocytes. In FIG. 26 it is shown that the application of 1 mg/ml of Peptide 2 (KKEKIG, SEQ ID NO:6) (FIG. 26 A) or of Peptide 3 (KKKRQ, SEQ ID NO:8) (FIG. 26 B) leads to a significant increase in the intracellular calcium concentration in cultivated primary astrocytes. On the other hand Peptides 4 and 5 cause no increase of Ca concentration in astrocytes.

These results show that the putative neuropeptides can modulate the intracellular calcium concentration in various ways. In any case they suggest that for drastic upward regulation of the intracellular calcium concentration by SDF-1γ following pre-incubation of the cells with the anti-CXCR4 antibody the C-terminal region of SDF-1γ, and not the molecular segment agreeing with the SDF-1α and/or SDF-10β chemokines, is responsible. This finding confirms the particular and specific function of the C-terminus of SDF-1γ and substantiates the results obtained with the complete SDF-1γ molecule.

### Literature

Altschul, S. F., Gish, W., Miller, W., Myers, E. W. and Lipman, D. J. (1990) Basic local alignment search tool. J. Mol. Biol. 215, 403-410.

Angerer, L. M., Stoler, M. H. and Angerer, R. C. (1987) In situ hybridization with RNA probes: An annotated recipe. In K. L. Valentino, R. H. Eberwine, J. D. Barchas (Hrsg.) In Situ Hybridization. Application to neurobiology, Oxford University Press, New York, 42-70.

Chomczynski, P. and Sacchi, N. (1987) Single step method of RNA isolation by acid guanidinium thiocyanate-phenol-chlorofrom extraction. Anal. Biochem. 162, 156-159.

Devereux, J., et al. (1984) Nucleic Acids Research 12 (12): 387.

Doranz, B. J., Orsini, M. J., Turner, J. D., Hoffman, T. L., Berson, J. F., Hoxie, J. A., Peiper, S. C., Brass, L. F. and Doms, R.W . (1999) Identification of CXCR4 domains that support coreceptor and chemokine receptor functions J. Virol. 73, 2752-2761.

Eipper, B., Stoffers, D. and Xu, R. (1992) The biosynthesis of neuropeptides: peptide alpha amidation. Annu. Rev. Neurosci. 15, 57-85.

Gellissen, G. (2000) Heterologous protein production in methylotrophic yeasts Appl. Microbiol. Biotechnol. 54:741-750.

Henikoff und Henikoff (1992) Proc. Natl. Acad. Sci. USA 89:10915-10919.

Köller, H., Trimborn, M., from Giesen, H. -J., Schroeter, M. and Arendt, G. (2001) TNFα reduces glutamate induced intracellular Ca²⁺increase in cultured cortical astrocytes. Brain Research 893:237-243.

Loetscher, P., Gong, J. H., Dewald, B., Baggiolini, M. and Clark-Lewis, I. (1998) N-terminal peptides of stromal cell-derived factor-1 with CXC chemokine receptor 4 agonist and antagonist activities. J. Biochem. 273 (35):22279-83.

Müiller, H. W., Ignatius, M. J., Hangen, D. H. and Shooter, E. M. (1986) Expression of specific sheath cell proteins during peripheral nerve regeneration in mammals, J. Cell Biol. 102, 393-402.

PCT1385-1966 35.

Needleman and Wunsch (1970) J. Mol. Biol.48:443-453.

Pearson, W. R. (1990) Rapid and sensitive sequence comparison with FASTP and FASTA. Methods Enzymol. 183, 63-98.

Rollins, B. J. (1997) Chemokines. Blood 90, 909-928.

Sambrook, J., Fritsch, E. F. and Maniatis, T. (1989) Molecular cloning: A laboratory Manual 2. Ausgabe, Cold Spring Harbor Laboratory Press, Cold Spring Harbor, N.Y.

Sanger, N., Nicklen, S. and Coulson, A. R., (1977) DNA sequencing with chain-terminating inhibitors. Proc. Natl. Acad. Sci. USA, 74, 144-148.

Shirozu, M., Nakano, T., Inazawa, J., Tashiro, K., Tada, H., Shinohara, T. and Honjo, T. (1995) Structure and chromosomal localization of the human stromal cell-derived factor 1 (SDF1) gene. Genomics 28, 495-500.

Siegel, G., Agranoff, B. Albers, R. W., Molinoff, P. (1989) Basic Neurochimstry, Raven Press, New York.

Stürzl, M., Roth, W. K., Viehweger, P. and Hoffschneider, H.Ü. (1991) Taq DNA-polymerase synthesized single stranded DNA hybridization probes and their application in Northern blotting and in-situ hybridization. In Rolfs, A.,

Schumacher, A. C., Marx, P. (Hrsg.) PCR-Topics. Usage of Polymerase Chain Reaction in Genetic and Infectious Diseases. Berlin, Springer-Verlag, 41-45.

Tashiro, K. Tada, H., Heilker, R., Sherozou, M., Nakano, T. and Honjo, T. (1993) Signal sequence trap: a cloning strategy for secreted proteins and type I membrane proteins. Science 261, 600-602.

Weydemann, U., Keup, P., Piontek, M., Strasser, A. W. M., Schweden, J., Gellissen, G., Janowicz, Z. A. (1995) High level secretion of hirudin by Hansenula polymorpha—authentic processing of three different preprohirudins. Appl. Microbiol. Biotechnol. 44: 844-849.

1. Nucleic acid molecule with a sequence comprising
(1) nucleic acid sequence coding for a chemokine, a neuropeptide precursor or at least one neuropeptide, which is selected from the following sequences:
(a) a nucleic acid sequence agreeing with SEQ ID NO: 1;
(b) a nucleic acid sequence which codes for a polypeptide with an amino-acid sequence agreeing with SEQ ID NO:2;
(c) a nucleic acid sequence which is at least 60% identical with the sequence indicated in (a);
(d) a sequence which hybridizes with the opposing strand of the sequence indicated in (a), or would be hybridized under conditions of degeneration of the genetic code;
(e) a derivative obtained through substitution, addition, inversion and/or deletion of one or more nucleotides of one or more of the sequences indicated in (a) or (b), which codes for a chemokine, a neuropeptide precursor or at least one neuropeptide; or
(2) a sequence complementary to one of the nucleic acid sequences given under (a) to (e).

2. Nucleic acid molecule in accordance with claim 1, characterized thereby that it is at least 80% identical with the nucleic acid sequence given under (1) (c)

3. Nucleic acid molecule in accordance with claim 1, characterized thereby that it is at least 90% identical with the nucleic acid sequence given under (1) (c).

4. Nucleic acid molecule in accordance with claim 1, characterized thereby that the nucleic acid sequence given under (1) (c) is at least 95% identical.

5. Nucleic acid molecule in accordance with claim 1, characterized thereby that it includes a nucleic acid sequence agreeing with SEQ ID NO:3.

6. Nucleic acid molecule in accordance with claims 1, characterized thereby that it includes a nucleic acid sequence which codes for a polypeptide with an amino acid sequence which agrees with SEQ ID NO:4.

7. Nucleic acid molecule in accordance with one of claims 1 to 6, comprising in addition a promotor suitable for expression, in which the coding nucleic acid sequence remains under the control of the promotor.

8. Nucleic acid molecule in accordance with one of claims 1 to 7, comprising in addition sequences of a vector which enables the replication of the nucleic acid molecule in a host cell and/or the integration of the nucleic acid molecule into the genome of a host cell.

9. Host cell containing a nucleic acid molecule in accordance with one of claims 1 to 8, by which the host cell is a prokaryotic or eukaryotic cell suitable for the expression of the nucleic acid molecule.

10. Host cell in accordance with claim 9, characterized thereby that the prokaryotic host cell is E. coli.

11. Host cell in accordance with claim 9, characterized thereby that the eukaryotic host cell is a fungal, an insect or a mammalian cell.

12. Host cell, in accordance with claim 11, characterized thereby that the host cell is the yeast Saccharomyces cerevisiae, the methylotrophic yeast Hansenula polymorpha, the dimorphic yeast Arxula adeninivorans or the filamentous fungus Sordaria macrospora.

13. Host cell in accordance with claim 11, characterized thereby that the host cell is a CHO, COS or HeLa cell.

14. Polypeptide molecule comprising an amino-acid sequence selected from the following sequences:
(i) an amino-acid sequence which includes an amino-acid sequence agreeing with SEQ ID NO:5, SEQ ID NO:6, SEQ ID NO:7, SEQ ID NO:8, SEQ ID NO:9 and/or SEQ ID NO:10; and/or a combination of, two or more of these sequences;
(ii) an amino-acid sequence agreeing with SEQ ID NO:4;
(iii) an amino-acid sequence corresponding to the sequence of amino-acid 20 to amino-acid 119 in SEQ ID NO:4;
(iv) an amino-acid sequence agreeing with SEQ ID NO:22;
(v) an amino-acid sequence which is at least 85% identical with the sequence given under (i), (ii), (iii) or (iv).

15. Polypeptide molecule in accordance with claim 14, characterized thereby, that the sequence given under (v) is at least 90% identical.

16. Polypeptide molecule in accordance with claim 14, characterized thereby, that the sequence given under (v) is at least 95% identical.

17. Polypeptide molecule in accordance with claim 14, characterized thereby, that it contains an amino-acid sequence agreeing with SEQ ID NO:12 or SEQ ID NO:13.

18. Fragment of a polypeptide molecule in accordance with one of the claims 14 to 17 which contains at least one neuropeptide.

19. Fragment in accordance with claim 18, comprising at least one of the amino-acid sequences agreeing with SEQ ID NO:5, SEQ ID NO:6, SEQ ID NO:7, SEQ ID NO:8, SEQ ID NO:9 and/or SEQ ID NO:10.

20. Procedure for the manufacture of a polypeptide molecule and/or of a fragment thereof in accordance with one of the claims 14 to 19, comprising the cultivation of a host cell in accordance with one of the claims 9 to 13 under suitable conditions for the expression and possible processing and if necessary the purification of the polypeptide molecules or fragments expressed.

21. An antibody which is specific for a polypeptide molecule and/or a fragment thereof in accordance with one of claims 14 to 19.

22. In vitro procedure for the demonstration in a biological specimen of a polypeptide molecule and/or a fragment thereof in accordance with one of claims 14 to 19, comprising bringing into contact of the specimen with a reagent specific for the polypeptide molecule and/or the fragment, and the demonstration of binding.

23. Test-kit for demonstration of a polypeptide and/or a fragment thereof in accordance with one of claims 14 to 19, comprising at least one reagent which is specific for the polypeptide and/or the fragment.

24. Test-kit in accordance with claim 23, characterized thereby, that it includes at least one antibody specific in accordance with claim 21 for the polypeptide molecule and/or the fragment.

25. In vitro procedure for the demonstration of a nucleic acid coding for a polypeptide molecule in accordance with one of claims 14 to 17 in a biological specimen, comprising:
the bringing into contact of the specimen with a nucleic acid molecule in accordance with one of claims 1 to 6 and/or a fragment thereof, of which the nucleic acid molecule and/or the fragment bears a demonstrable token, and
the demonstration of the token.

26. Pharmaceutical preparation containing at least one polypeptide molecule and/or a fragment thereof in accordance with one of claims 14 to 19, and/or a pharmaceutically compatible salt of such a molecule or fragment.

27. Pharmaceutical preparation in accordance with claim 26, characterized thereby that it contains in addition at least one antibody.

28. Pharmaceutical preparation in accordance with claim 26 or 27, for therapeutic use in demyelinating or neurodegenerative diseases or in developmental disorders of the nervous system.

29. Pharmaceutical preparation in accordance with claim 26 or 27, for the prevention or treatment of an HIV infection and in particular an HIV encephalopathy in humans.

30. Pharmaceutical preparation in accordance with claim 26 or 27, for therapeutic use in diseases of the haemopoietic system, the immune system and the cardiovascular system.

31. Pharmaceutical preparation containing at least one reagent specific for a polypeptide molecule and/or a fragment thereof in accordance with one of the claims 14 to 19.

32. Pharmaceutical preparation in accordance with claim 31, characterized thereby, that the reagent is an antibody.

33. Pharmaceutical preparation containing at least one nucleic acid molecule in accordance with one of the claims 1 to 8.

34. Pharmaceutical preparation in accordance with one of claims 31 to 33 for diagnostic or therapeutic use in demyelinating or neurodegenerative diseases or in developmental disorders of the nervous system.

35. Pharmaceutical preparation in accordance with one of claims 31 to 33 for the diagnosis or treatment of an HIV infection and in particular an HIV encephalopathy in humans.


SEQUENCE LISTING (22 sequences)

SEQ ID NO: 1 - DNA, 90 bp, Artificial sequence
  Description of the artificial sequence consensus sequence for the specific region of SDF-1-gamma
gggcgcagagaagaaaaagtggggaaaaaagaaaagataggaaaaaagaagcgacagaagaagagaaaggckgcccagaaaargaaaaac

SEQ ID NO: 2 - protein, 30 aa, Artificial sequence
  Description of the artificial sequence consensus sequence for the specific region of SDF-1-gamma
GRREEKVGKKEKIGKKKRQKKRKAAQKXKN

SEQ ID NO: 3 - DNA, 360 bp, Artificial sequence
  Description of the artificial sequence consensus sequence for the specific region of SDF-1-gamma
atgracgccaaggtcgtsgycgtgctggycctsgtgctgrccgcgctctgcmtcagygacggkaagccmgtcagcctgagctacagatgcccmtgccgattcttygaragccatgtygccagagccaacgtcaarcatctsaaaatyctcaacactccaaactgtgcccttcagattgtwgcmmggctgaararcaacaacagacaagtgtgcattgacccgaarytaaagtggatycargagtacctggasaaagcyttaaacaaggggcgcagagaagaaaaagtggggaaaaaagaaaagataggaaaaaagaagcgacagaagaagagaaaggckgcccagaaaargaaaaactag

SEQ ID NO: 4 - protein, 119 aa, Artificial sequence
  Description of the artificial sequence consensus sequence for the SDF-1-gamma-Polypeptide.
MBAKVVXVLXLVLXALCXSDGKPVSLSYRCPCRFFESHVARANVKHLKILNTPNCALQIVARLKXNNRQVCIDPKLKWIQEYLXKALNKGRREEKVGKKEKIGKKKRQKKRKAAQKXKN

SEQ ID NO: 5 - protein, 7 aa, Artificial sequence
  Description of the artificial sequencepeptide obtainable through proteolytic splitting of SDF-1-gamma
RREEKVG

SEQ ID NO: 6 - protein, 6 aa, Artificial sequence
  Description of the artificial sequencepeptide obtainable through proteolytic splitting of SDF-1-gamma
KKEKIG

SEQ ID NO: 7 - protein, 90 aa, Artificial sequence
  Description of the artificial sequencepeptide obtainable through proteolytic splitting of SDF-1-gamma
MBAKVVXVLXLVLXALCXSDGKPVSLSYRCPCRFFESHVARANVKHLKILNTPNCALQIVARLKXNNRQVCIDPKLKWIQEYLXKALNKG

SEQ ID NO: 8 - protein, 5 aa, Artificial sequence
  Description of the artificial sequencepeptide obtainable through proteolytic splitting of SDF-1-gamma
KKKRQ

SEQ ID NO: 9 - protein, 7 aa, Artificial sequence
  Description of the artificial sequencepeptide obtainable through proteolytic splitting of SDF-1-gamma
KKRKAAQ

SEQ ID NO: 10 - protein, 4 aa, Artificial sequence
  Description of the artificial sequencepeptide obtainable through proteolytic splitting of SDF-1-gamma
KXKN

SEQ ID NO: 11 - DNA, 5391 bp, Rattus norvegicus
  misc_feature (3841)..(3841): n is a, c, g, or t
tgtcctcttgctgtccagctctgcagcctccggcgcgccctcccgcccacgccatggacgccaaggtcgtcgccgtgctggccctggtgctggccgcgctctgcatcagtgacggtaagccagtcagcctgagctacagatgcccctgccgattctttgagagccatgtcgccagagccaacgtcaaacatctgaaaatcctcaacactccaaactgtgcccttcagattgttgcaaggctgaaaagcaacaacagacaagtgtgcattgacccgaaattaaagtggatccaagagtacctggacaaagccttaaacaaggggcgcagagaagaaaaagtggggaaaaaagaaaagataggaaaaaagaagcgacagaagaagagaaaggcggcccagaaaaagaaaaactagttacgtgcttcctgcagatggaccacagtacgctctgctctggcgctttgtaacccccccttccctctccgggggcagaccccacactccgggcaggtgctcagactgatggtaaactcttccctcttctgggggcagaccacacatcccagggaagaccccacacccccgggcagatgcttaggctttcctgccccggcggccacaccagctgctgtatttacgcgcttcttaaggccctgctctgtctgctaagctatgaagaaagatgtgcagagactggggtggaggctaagccacagaggacctgcctagcctggcagcttgccccgagctgagccccttggccaggagttcacaaggctcacacctacaatcccatgaaggccagggtggtctgcttagccaggaaagggcatgtgccttcccctcaaccacactgccccctgtggccttctcaggtaactgacttgctctcaggcccacgggaagcttttccaaatacctgcggcctgggaagggacttcattcagccctgctgcccgggctgtgggagcagcttggtttcaacacagaagggtatctgcagactgtgttgggtgaaaagcaggaagaatgaagtctcagagaacgcatgttagctgcttctcagggaatctttcctttggaaaattcactttagagtctttaaacgggtccctcatggggagggcagatgtgctctgggactttctgatgggccagcagcttcagggactcttagtctgtcctccccacccttggtctcaacattcccaggatggtgtgctatccggtcaccaatgcctccgtcctcactcctgagagatgtctgccttctgtggattgggttaaagctctggaattacctaatatcccaacccaccaccctcacctggcaatttttgtctagtcttttgtttttgtctttctccattttggattagaaggatagagggcaaggctctgattttagcagtgttttggagaaaaaattttttttcttcatctcatgtagacacacacacacacacacacacacacacacacacacacacacacacacacatcttgtaccccagacctctgggtctaattttcataattggggcagaaagaagaaatgatctgaagatacagcaaatggattgcaggggaaggaaggcccagtgtcctgtgtgtcatgccctcttgggtccctaagttctaggttccttagagggtctaacattaaataggtaagaggccttcatggtccttgctggggaagggtctcaccagggagcttcagggaagacccatgttacgaactcttatgctttatctggacagccctcctggtccataccctctcctcagatctgaggtagcggggtgggctattggtgggcgtctttcaagcccagggttactgtctgttcttcttggggcagccagttacagtctggtctcagtggccttggctgcatccttcctactgttgacaaaacacttctgaaggccagatctgtgcccaagccatagttctgcctagaaatggatgcccagcccctccaggacactgggaaggactgttggcccctaacaaccaaaggccatactgaggctgccctgagttggaagaccactttccgaaatgcccctggactctgcctcccaccatccacccctgactcctaggagttagagagtaggaaaacagtttgtttcttaggaaccacagcaagctcccaggagccctctgtgcttatgaagcccatctaatgggcagccccagccttctggacagagtcctcatggaaatgcgtgagaagctgatttcgtctaaggatgggttgaaggtaggatgtgctcctgtatgttctcaggcaggtgagagagggtcttccttacagtatctagcataaacaccttctggaaggttctgcagctctagagatcacctcctcagtgccaagacctcttctggtggtgtgggagcagccaagagatttcaaggaagagtgattatttgatgaaataacttgaattatatcaagagtgaatatttgatgggaactgcctcttctcttggagttctgaggcctggggatgcccaggaactcagggcacctgctgttgttggagtcgatgcatagtctcaacaccagtgtcctaaggttaaggcagtgtgccttgtcatgtgttccttgtaccatgcctcctgtgccagtgtgtgtgccttagcctgtgcttgacatgttcacccgtcttctctgcttgccaccaccacccagaccctcagcatcagtcctggctgtgcccctccctgccctcccactctctcaggccttggaaggaagatggctcgactgcaagctgaactaaggagtagggcctgtggctcctgccaggccacacagcatcccaggcacgtggtgagaatccgccttaatgtgtctcctctgttcttgtcaacaggaggctcaagatgtgagaggtgtgagtcagacgcctgaggaacttacagaaggagcctaggtctgaagtcagtgttagggaagggcccatagccacttcctctgctcctgagcagggctgaagccatttccaagggacttgctttgcagtttgctacactttcaccatttgattatatagcaagatacatggtaattattttattttcatttagtctgattctccaatgtcattggtgacaggccaaggccactatgtcatctcctttgttctagtatctttcccatgaaggaccttttctgaatagtggctcccaaggtttgtctctttgagctgaggcaggaggctcacctttttctgattagaaactgggtgttcctacccccaaggattgcagggctttccccaagctgaggcaggagtgtgaggtcagggaagagcgagatccaccctcatcccatgctctcctcttcatcccaccatgctcatctctgtctcatccatcaccgtgtgtctgcaagactgtctccatgacccggaaaaaggactctctcaagaggaactcctttactcaaaatgggacagcaagaaggaaaaggaagtgtctgttgttccgcccaaacccttcgcgcgtytattgtcttgtttggaatattgtctcttcaaccccctgcttctgttgacctccatgaccaatgtctcgtctgtgcactgtctctaacccaaatgcaaaggctgtgtatgaggtaatggccctgaggtccaggttttcatggaaacagcgcactgtctccctgttcacaggctcattttggacacacagagcccaaagaaaggtggtttgcaacagagctcagctctaagactgtagatccttcatattttcgtactgttasstttaaattgtgggttcttassttcctggaaccgaatgcattcttctattgagnactagcaggtctcagttctttccaattatttttaaaagccaatgaataaaagcatcagcattgggcccactgggcgggcatttctctagaaaggggagaaccacctacctttccttaggacagccgaccagcacggncccaggaagtgnnnnnntcttctgcagtttttatacaagctcccctgccacctttgacaaacgcacagttaagagtcagtatctagttcttcagagacaaagatggagggagtaagaaggggaagggaaacggagaaagctaccaaaagatcatcctcaaaagcnggtgtttgagagtgaacgagctgtagaattgttagtgatgtgtgtgtggtgagggatttctataaatagtcattcaagttgatttcacagcagatgaaaaatccaaccagcaagattttgatcaaatttggacaacagcaacaaawctaaaaatgtgaagccagttgggataaggggcatatggtttgctgcagactgggtccccatgtggattcagaattattttaaactctcttgacatccggggcccccacaagagaaatctggattgctgtgcaatggccacttagcatctaatccaagctttgaaggaaacaaatacagccttgcaccttcnctccagttagggatccttttaaagtctccttcacagggaggataaagagactgggtagaaactggagggagatgaatttgtgtatcaattccgctgctgacagtcattttctagawggagacagcctgcctagagcaaatgtgcanttwaataggrcatttacatnggrarmgcctctccccaccttnatcccccatgctcttrctttcaaaatnacaagncacagcagtccttgaatggttgttgacsccgsacacctaactgtccctgatgatcctggtgcwgcccngaattcccttggncgccaagtaacctgccaggcagccnagtccctytgtcaccagcctttgcatctggatagggaaaagggggttggagacatacagtctgctttgtgttgaanccnagattngtacsctgtgtttacactgtgctgcctgctctcgggnacagtgggaaggaagtgcagccgaggtggcagacccctctgattcattsctggtcggctttgagggagggtttggagagcaaaaggctgcattcctctgtgggacttgcctgagcctttagsyctctccatcgagttctgtttatcttctcatgggtgattatctcggcggcgtcaccaggggcttcctcacagaagtcatncctcngcagagcttgcagtgtctacgcagcgatggtttcagtgttgcatgtggtgaatactgtattttgtttcagttctgtctcccagataatgtgaaaatggtccaggagaaggcagcttcctatacgcagtgtgtgctttcttattctcgtttttaatatatgacagttatttgagaggccatttctactttgaagtcatatcaatgaaaatgatgtatcttcacctacaatttttcctaataaagttctgtattcga

SEQ ID NO: 12 - protein, 119 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 13 - protein, 119 aa, Rattus norvegicus
MDAKVVAVLALVLAALCISDGKPVSLSYRCPCRFFESHVARANVKHLKILNTPNCALQIVARLKSNNRQVCIDPKLKWIQEYLDKALNKGRREEKVGKKEKIGKKKRQKKRKAAQKKKN

SEQ ID NO: 14 - DNA, 360 bp, Homo sapiens
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaaggggcgcagagaagaaaaagtggggaaaaaagaaaagataggaaaaaagaagcgacagaagaagagaaaggctgcccagaaaaggaaaaactag

SEQ ID NO: 15 - DNA, 360 bp, Rattus norvegicus
atggacgccaaggtcgtcgccgtgctggccctggtgctggccgcgctctgcatcagtgacggtaagccagtcagcctgagctacagatgcccctgccgattctttgagagccatgtcgccagagccaacgtcaaacatctgaaaatcctcaacactccaaactgtgcccttcagattgttgcaaggctgaaaagcaacaacagacaagtgtgcattgacccgaaattaaagtggatccaagagtacctggacaaagccttaaacaaggggcgcagagaagaaaaagtggggaaaaaagaaaagataggaaaaaagaagcgacagaagaagagaaaggcggcccagaaaaagaaaaactag

SEQ ID NO: 16 - DNA, 2819 bp, Rattus norvegicus
  misc_feature (1269)..(1269): n is a, c, g, or t
tgtcctcttgctgtccagctctgcagcctccggcgcgccctcccgcccacgccatggacgccaaggtcgtcgccgtgctggccctggtgctggccgcgctctgcatcagtgacggtaagccagtcagcctgagctacagatgcccctgccgattctttgagagccatgtcgccagagccaacgtcaaacatctgaaaatcctcaacactccaaactgtgcccttcagattgttgcaaggctgaaaagcaacaacagacaagtgtgcattgacccgaaattaaagtggatccaagagtacctggacaaagccttaaacaagaggctcaagatgtgagaggtgtgagtcagacgcctgaggaacttacagaaggagcctaggtctgaagtcagtgttagggaagggcccatagccacttcctctgctcctgagcagggctgaagccatttccaagggacttgctttgcagtttgctacactttcaccatttgattatatagcaagatacatggtaattattttattttcatttagtctgattctccaatgtcattggtgacaggccaaggccactatgtcatctcctttgttctagtatctttcccatgaaggaccttttctgaatagtggctcccaaggtttgtctctttgagctgaggcaggaggctcacctttttctgattagaaactgggtgttcctacccccaaggattgcagggctttccccaagctgaggcaggagtgtgaggtcagggaagagcgagatccaccctcatcccatgctctcctcttcatcccaccatgctcatctctgtctcatccatcaccgtgtgtctgcaagactgtctccatgacccggaaaaaggactctctcaagaggaactcctttactcaaaatgggacagcaagaaggaaaaggaagtgtctgttgttccgcccaaacccttcgcgcgtytattgtcttgtttggaatattgtctcttcaaccccctgcttctgttgacctccatgaccaatgtctcgtctgtgcactgtctctaacccaaatgcaaaggctgtgtatgaggtaatggccctgaggtccaggttttcatggaaacagcgcactgtctccctgttcacaggctcattttggacacacagagcccaaagaaaggtggtttgcaacagagctcagctctaagactgtagatccttcatattttcgtactgttasstttaaattgtgggttcttassttcctggaaccgaatgcattcttctattgagnactagcaggtctcagttctttccaattatttttaaaagccaatgaataaaagcatcagcattgggcccactgggcgggcatttctctagaaaggggagaaccacctacctttccttaggacagccgaccagcacggncccaggaagtgnnnnnntcttctgcagtttttatacaagctcccctgccacctttgacaaacgcacagttaagagtcagtatctagttcttcagagacaaagatggagggagtaagaaggggaagggaaacggagaaagctaccaaaagatcatcctcaaaagcnggtgtttgagagtgaacgagctgtagaattgttagtgatgtgtgtgtggtgagggatttctataaatagtcattcaagttgatttcacagcagatgaaaaatccaaccagcaagattttgatcaaatttggacaacagcaacaaawctaaaaatgtgaagccagttgggataaggggcatatggtttgctgcagactgggtccccatgtggattcagaattattttaaactctcttgacatccggggcccccacaagagaaatctggattgctgtgcaatggccacttagcatctaatccaagctttgaaggaaacaaatacagccttgcaccttcnctccagttagggatccttttaaagtctccttcacagggaggataaagagactgggtagaaactggagggagatgaatttgtgtatcaattccgctgctgacagtcattttctagawggagacagcctgcctagagcaaatgtgcanttwaataggrcatttacatnggrarmgcctctccccaccttnatcccccatgctcttrctttcaaaatnacaagncacagcagtccttgaatggttgttgacsccgsacacctaactgtccctgatgatcctggtgcwgcccngaattcccttggncgccaagtaacctgccaggcagccnagtccctytgtcaccagcctttgcatctggatagggaaaagggggttggagacatacagtctgctttgtgttgaanccnagattngtacsctgtgtttacactgtgctgcctgctctcgggnacagtgggaaggaagtgcagccgaggtggcagacccctctgattcattsctggtcggctttgagggagggtttggagagcaaaaggctgcattcctctgtgggacttgcctgagcctttagsyctctccatcgagttctgtttatcttctcatgggtgattatctcggcggcgtcaccaggggcttcctcacagaagtcatncctcngcagagcttgcagtgtctacgcagcgatggtttcagtgttgcatgtggtgaatactgtattttgtttcagttctgtctcccagataatgtgaaaatggtccaggagaaggcagcttcctatacgcagtgtgtgctttcttattctcgtttttaatatatgacagttatttgagaggccatttctactttgaagtcatatcaatgaaaatgatgtatcttcacctacaatttttcctaataaagttctgtattcga

SEQ ID NO: 17 - protein, 93 aa, Rattus norvegicus
MDAKVVAVLALVLAALCISDGKPVSLSYRCPCRFFESHVARANVKHLKILNTPNCALQIVARLKSNNRQVCIDPKLKWIQEYLDKALNKRLKM

SEQ ID NO: 18 - protein, 5 aa, Artificial sequence
  Description of the artificial sequence peptide obtainable through proteolytic splitting of SDF-1-beta
KRLKM

SEQ ID NO: 19 - DNA, 21 bp, Artificial sequence
  Description of the artificial sequence Primer MMSE2
acgccatggacgccaaggtcg

SEQ ID NO: 20 - DNA, 25 bp, Artificial sequence
  Description of the artificial sequence Primer GAS2
actgtaaggaagaccctctctcacc

SEQ ID NO: 21 - DNA, 25 bp, Artificial sequence
  Description of the artificial sequence Primer GAS3
gttgagactatgcatcgactccaac

SEQ ID NO: 22 - protein, 101 aa, Artificial sequence
  Description of the artificial sequence amino-acid sequence of the mature human SDF-1-gamma protein with a C-terminal methionine(construct M-mhSDF-1-gamma)
MDGKPVSLSYRCPCRFFESHVARANVKHLKILNTPNCALQIVARLKNNNRQVCIDPKLKWIQEYLEKALNKGRREEKVGKKEKIGKKKRQKKRKAAQKRKN